(19) United States
(12) Patent Application Publication
(10) Pub. No.: US 2009/0105806 A1
(43) Pub. Date: Apr. 23, 2009

(54) STENT

(75) Inventors: Joshua Benjamin, Aliso Viejo, CA (US); Stefan Schreck, Fallbrook, CA (US)

Correspondence Address: KNOBBE MARTENS OLSON & BEAR LLP, 2040 MAIN STREET, FOURTEENTH FLOOR, IRVINE, CA, 92614, US

(73) Assignee: ENDOLOGIX, INC, Irvine, CA (US)

(21) Appl. No.: 12/257,149

(22) Filed: Oct. 23, 2008

Related U.S. Application Data
(60) Provisional application No. 60/981,869, filed on Oct. 23, 2007.

(51) Int. Cl. A61F 2/06

(52) U.S. Cl. 623/1.15; 623/1.16; 623/1.13

(57) ABSTRACT

A tubular prosthesis can be formed by rolling a sheet of material about a longitudinal axis, thereby forming a plurality of annular rings that can be arranged so as to be longitudinally adjacent one another, thus forming a tubular prosthesis. Each annular member can expand and contract independent of the other annular members. A graft material can extend between the plurality of annular members.

### PRIORITY INFORMATION

This application also claims priority benefit under 35 U.S.C. § 119(e) of Provisional Application 60/981,869 filed Oct. 23, 2007, which application is hereby incorporated by reference as if fully set forth herein.

### BACKGROUND OF THE INVENTION

1. Field of the Invention

The present invention relates to stents. More specifically, this invention relates to a flexible stent generally usable for peripheral angioplasty.

2. Description of the Related Art

Stents are typically used in a number of medical procedures such as coronary or peripheral angioplasty procedures. The term coronary angioplasty generally refers to a medical procedure in which a balloon or other mechanical device can be used to open a blockage in a coronary artery narrowed by atherosclerosis, thus improving the blood flow to the heart. The term peripheral angioplasty generally refers to the use of mechanical devices for widening an opening in blood vessels other than the coronary arteries, such as femoral arteries. Peripheral angioplasty is often called percutaneous transluminal angioplasty (“PTA”). PTA can be most commonly done to treat narrow portions in the leg arteries, especially the common iliac, external iliac, superficial femoral, and popliteal arteries. PTA can also be done to treat narrow regions in veins. Flexible stents are particularly suited for peripheral angioplasty because the stent is typically longer and subject to increased bending stresses as the user's leg is rotated about the hip joint.

Stents used for peripheral angioplasty typically take the form of radially expandable, cylindrical or tubular shaped prostheses that can be highly flexible so as to not restrict the free movement of the femoral artery or other vessel or vein in which the stent is placed. Such stents are typically introduced via a catheter assembly into a lumen of a body passage in a restricted state. Thereafter, the stents typically radially expand, thus expanding the internal diameter of the blood vessel, artery, vein, or other body passage into which it is placed. Generally, the degree of expansion is governed by several factors, including, but not limited to, the internal diameter of the blood vessel, artery, vein, or other body passage into which the stent has been positioned. As such, the stents thus provide support to sections of vessel walls such as arteries or other body passages that have collapsed, are partially occluded, blocked, weakened, or dilated, and the stents can maintain these body passages in a generally open, unobstructed state, enabling the flow of blood through such vessel to substantially return to its previous, unobstructed level.

### SUMMARY OF THE INVENTION

An aspect of some embodiments is a generally flexible, cylindrical stent configured to support or expand the inside of a body passage. The stent can comprise a plurality of annular members generally arranged in series. Each annular member can comprise an opening disposed generally axially therethrough to allow the flow blood therethrough. In some embodiments, each annular member can define a space therebetween. Each annular member can comprise an exterior portion and an overlapping portion. The exterior portion can be configured to form a complete annulus and the overlapping portion generally arranged to be helically positioned adjacent to an inside surface of the exterior portion such that an end portion of the overlapping portion can be generally positioned adjacent to the inside surface of the overlapping portion when the stent is in a pre-placement or post-placement state. Each annular member can be configured to expand and contract independent of the other annular members of the plurality of annular members.

Some embodiments of the present disclosure are directed to a prosthesis configured to support or expand a body passage. In some embodiments, the prosthesis can comprise a plurality of annular members and a graft extending between the plurality of annular members. The annular members can be arranged in series along a longitudinal axis of the prosthesis such that each of the annular members is longitudinally adjacent another annular member. Each annular member can have a generally annular shape about the longitudinal axis. Each annular member can define a first end and a second opposite end, an inside surface extending between the first end and the second end, and an outside surface extending between the first end and the second end. Each annular member can be wrapped around the longitudinal axis to form an overlapping portion where a portion of the inside surface of the annular member can be adjacent to a portion of the outside surface of the respective annular member to define a longitudinal lumen having openings at a distal end of the prosthesis and at a proximal end of the prosthesis. Each annular member can be configured to expand and contract independent of the other annular members as the first end of an annular member moves towards the second end of the annular member and a length of the overlapping portion decreases.

In some embodiments, the prosthesis can be configured to be substantially cylindrically shaped in a relaxed state. In some embodiments, the prosthesis can be configured such that one or more of the annular members are formed from a generally flat sheet of material. In some embodiments, the prosthesis can be configured to comprise a means to secure one or more of the annular members to the graft, which can be, without limitation, adhesive, sutures, or other suitable attachment means. In some embodiments, the prosthesis can be configured such that the graft is formed from a generally flat sheet of material. In some embodiments, the prosthesis can be configured such that the graft is configured to cover only a portion of one or more of the annular members. The prosthesis can be configured such that the graft can be positioned adjacent to at least a portion of the outside surface and/or inside surface of one or more of the annular members. Alternatively, the prosthesis can be configured such that one or more of the annular members is embedded within the material forming the graft or positioned between two layers of the material forming the graft.

The prosthesis can be configured to comprise one or more connection members configured to interconnect one or more of the plurality of annular members. The prosthesis can be configured such that one or more of the annular members are formed from a material selected from the group consisting of: Nitinol, stainless steel, titanium, aluminum, gold, nickel-titanium, cobalt-chromium, cobalt-chromium-molybdenum, platinum, platinum-tungsten, platinum-nickel, and platinum-rhenium. The prosthesis can be configured such that the graft is formed from a material selected from the group consisting of: urethane, Teflon, PTFE, ePTFE, polyurethane, silicone elastomers, and polytetrafluoroethylene. The prosthesis can be configured such that the prosthesis comprises a coating on at least one or more surfaces of the annular members. The prosthesis can be configured such that the coating comprises at least one material selected from the group consisting of: polyurethane, silicone elastomer, polytetrafluoroethylene, nylon, and Teflon. The prosthesis can be configured such that one or more of the plurality of annular members is configured to promote tissue growth into at least a portion thereof. The prosthesis can be configured such that one or more of the plurality of annular members is configured to be selectively biased at one or more desired diameters.

Some embodiment of the present disclosure are directed to a method of making a prosthesis configured to support or expand a body passage. In some embodiments, the method can comprise (in no particular order): providing a first generally flat sheet of material suitable for a graft; providing a plurality of flat strips of material suitable for a stent; positioning the plurality of strips in a lengthwise orientation so that each of the strips is generally parallel to one another; and rolling (or otherwise processing) the strips simultaneously with the first sheet so that each strip and the first sheet form an approximately cylindrically shaped prosthesis in which a portion of each of the strips overlap each other to define an axially extending lumen.

In some embodiments, the strips of material can be interconnected or can be attached to the first sheet, with or without the use of adhesive. In some embodiments, one or more of the strips can define a space therebetween and can be attached to the first sheet using one or more sutures, or can be embedded within the first sheet. In some embodiments, the method can further comprise providing a second generally flat sheet of material suitable for a graft and melting the second sheet to the first sheet with strips being positioned generally between the first and second sheets. In some embodiments, the method can further comprise positioning the flat sheet adjacent to a first side of each of the strips, providing a second generally flat sheet of material suitable for a graft, positioning the second sheet adjacent to a second side of each of the strips, the second side opposing the first side, and then forming the strips simultaneously with the first and second sheets so that each strip and the first and second sheets form an approximately cylindrically shaped prosthesis. In some embodiments, the one or more of the strips can be formed to define an inside surface, an outside surface, and an overlapping portion, and the inside surface of the overlapping portion of the one or more formed strips can be adjacent to the outside surface of the respective annular member. In some embodiments, one or more of the plurality of annular members can be configured to promote tissue growth into at least a portion thereof.

### BRIEF DESCRIPTION OF THE DRAWINGS

These and other features, aspects and advantages of the present invention will now be described in connection with preferred embodiments of the invention, in reference to the accompanying drawings. The illustrated embodiments, however, are merely examples and are not intended to limit the invention. The following are brief descriptions of the drawings, which may not be drawn to scale.

FIG. 1 is a perspective view of a stent according to an embodiment of the present invention.

FIG. 2 is a side view of the embodiment of the stent shown in FIG. 1.

FIG. 3 is an enlarged end view of the embodiment of the stent shown in FIG. 1.

FIG. 4 is a top view of the embodiment of the stent shown in FIG. 1 in a partially formed state.

FIG. 5 is a partially sectioned perspective view of another embodiment of a stent positioned within a body passage.

FIG. 6 is a side view of the embodiment of the stent shown in FIG. 5 (body passage not illustrated).

FIG. 7A is an enlarged section view of the embodiment of the stent shown in FIG. 5 taken along line 7A-7A in FIG. 6 (body passage not illustrated).

FIG. 7B is an enlarged section view of the embodiment of the stent shown in FIG. 5 taken along line 7B-7B in FIG. 6 (body passage not illustrated).

FIG. 7C is an enlarged section view of the embodiment of the stent shown in FIG. 5 taken along line 7C-7C in FIG. 6 (body passage not illustrated).

FIG. 8 is a top view of the embodiment of the stent shown in FIG. 5 in a partially formed state.

FIG. 9 is a perspective view of another embodiment of a stent.

FIG. 10 is a side view of the embodiment of the stent shown in FIG. 9.

FIG. 11 is an enlarged section view of the embodiment of the stent shown in FIG. 9 taken along line 11-11 in FIG. 10.

FIG. 12 is a perspective view of another embodiment of a stent.

FIG. 13 is a side view of the embodiment of the stent shown in FIG. 12.

FIG. 14 is a section view of the embodiment of the stent shown in FIG. 12 taken along line 14-14 in FIG. 13.

FIG. 15 is a perspective view of another embodiment of a stent.

FIG. 16 is a side view of the embodiment of the stent shown in FIG. 15.

FIG. 17 is an enlarged section view of the embodiment of the stent shown in FIG. 15 taken along line 17-17 in FIG. 16.

FIG. 18 is a perspective view of another embodiment of a stent.

FIG. 19 is a perspective view of another embodiment of a stent.

FIG. 20 is an enlarged end view of the embodiment of the stent shown in FIG. 19.

FIG. 20A is a further enlarged end view of the embodiment of the stent shown in FIG. 19 taken along curve 20A-20A in FIG. 20.

FIG. 21 is an end view of another embodiment of a stent.

FIG. 21A is an enlarged end view of the embodiment of the stent shown in FIG. 20 taken along curve 21A-21A in FIG. 21.

FIG. 22 is a partially sectioned perspective view of another embodiment of a stent.

FIG. 23 is a section view of the embodiment of the stent shown in FIG. 22, showing the stent positioned within a body passage.

FIG. 24 is an enlarged end view of the embodiment of the stent shown in FIG. 22 (body passage not illustrated).

FIG. 25 is a top view of another embodiment of a stent, showing the stent in a partially formed state.

FIG. 26 is an enlarged end view of the embodiment of the stent shown in FIG. 25, showing the stent in a formed state.

FIG. 27 is a top view of another embodiment of a stent, showing the stent in a partially formed state.

FIG. 28 is an enlarged end view of the embodiment of the stent shown in FIG. 27, showing the stent in a formed state.

FIG. 29 is a top view of another embodiment of a stent, showing the stent in a partially formed state.

FIG. 30 is an enlarged end view of the embodiment of the stent shown in FIG. 29, showing the stent in a formed state.

FIG. 31 is a top view of another embodiment of a stent, showing the stent in a partially formed state.

FIG. 32 is an enlarged section view of the embodiment of the stent shown in FIG. 31 taken along line 32-32 in FIG. 31, showing the stent in a formed state.

### DETAILED DESCRIPTION OF THE PREFERRED EMBODIMENTS

The following detailed description is now directed to certain specific embodiments of the invention. In this description, reference is made to the drawings wherein like parts are designated with like numerals throughout the description and the drawings.

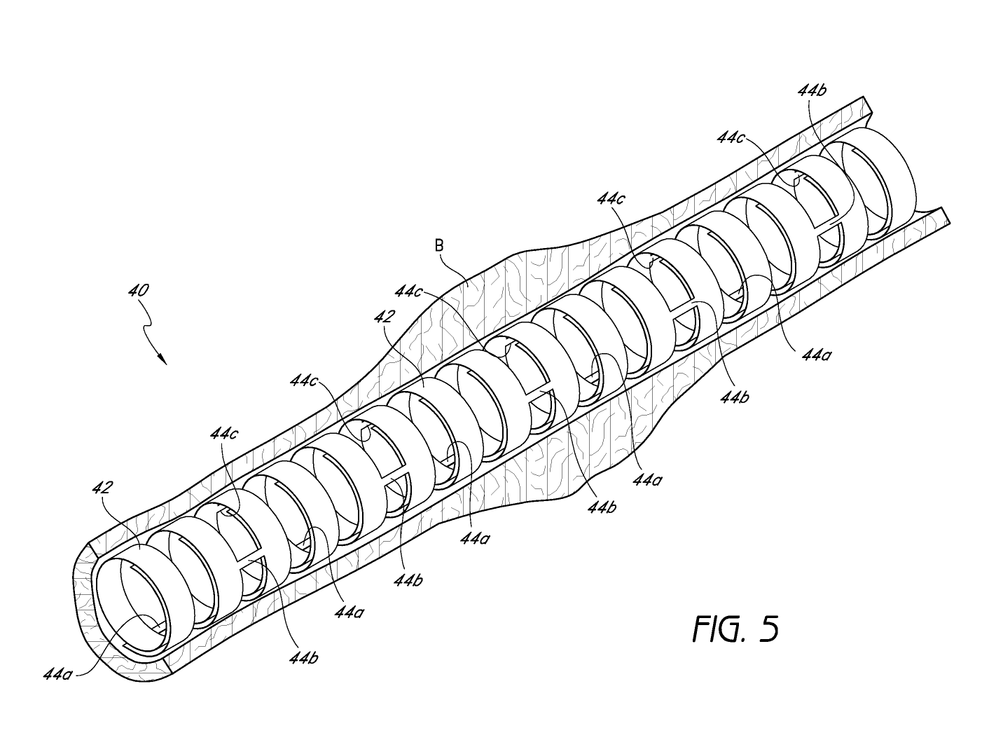

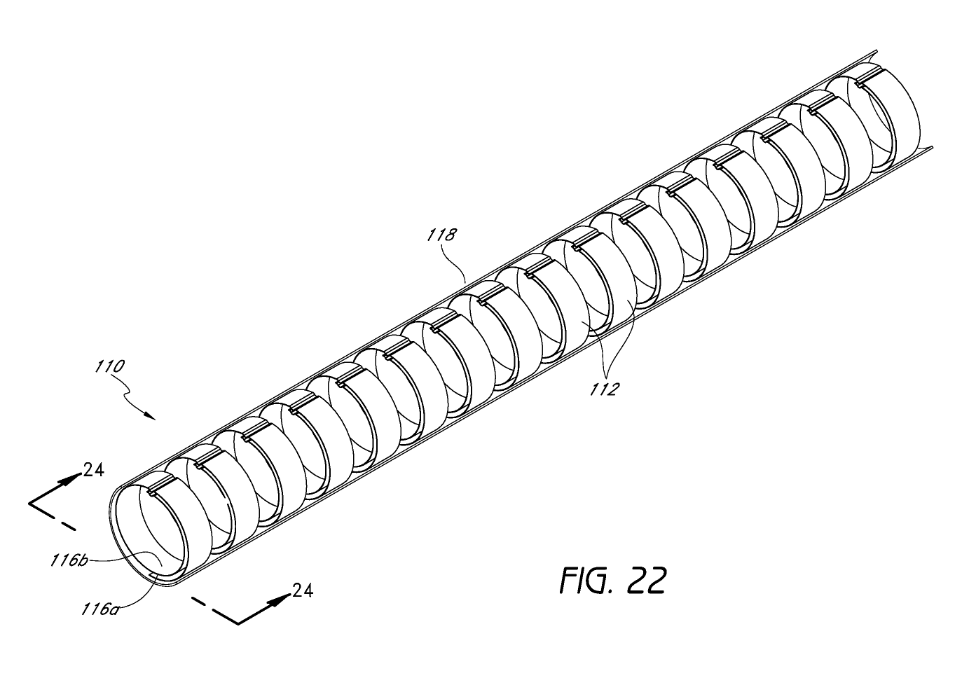

The embodiments of stents described and illustrated herein can be comprised of a plurality of independently expandable and contractible annular rings or members, which can be arranged in series. In some embodiments, the annular members can be interconnected by connecting segments (see FIG. 5) that can be integrally formed with the annular members. In some embodiments, the stent can be formed without such connecting segments (see FIGS. 22-23). The annular members can be configured such that, after the stent is positioned in the desired body passage, the annular members self-expand or such that the annular members can be mechanically expanded (e.g., via a balloon) within the body passage. It is to be understood that any reference to a body passage herein refers collectively to arteries, veins, or other passageways, blood vessels, or non-vascular body lumens, passages or chambers within the body of a human or animal.

The embodiments of the stents described and illustrated herein can be configured to be axially flexible so as to not significantly affect the movement of the body passage into which the stent is positioned, while maintaining a desired dilation of the body passage into which the stent is positioned. The embodiments of stents described and illustrated herein can be configured such that, when implanted into an occluded or otherwise obstructed body passage, the outer surface of the stent supports and expands the inner surface of the occluded or otherwise obstructed portion of the body passage. The inner aperture of the stent can allow blood or other body fluid to flow therethrough without substantial obstruction.

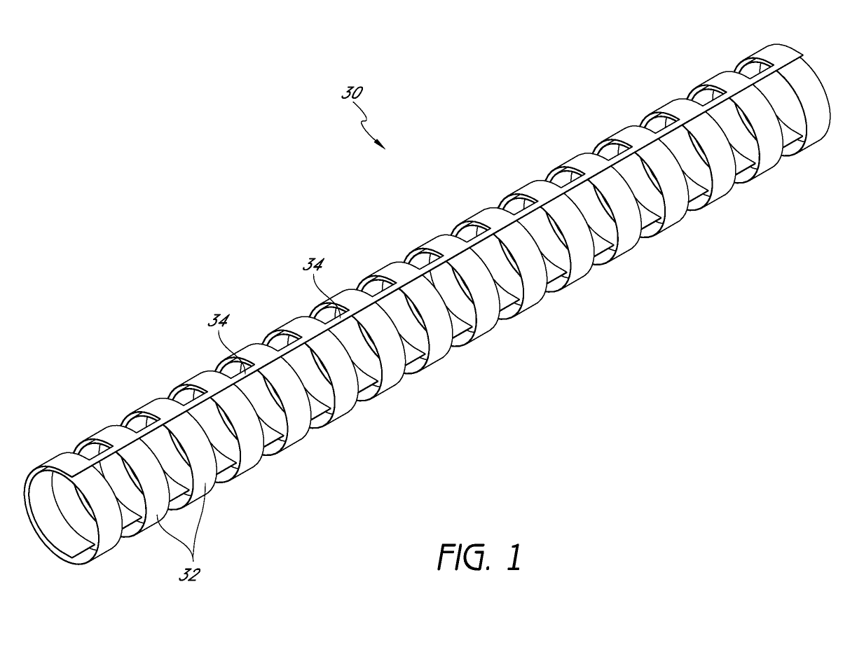

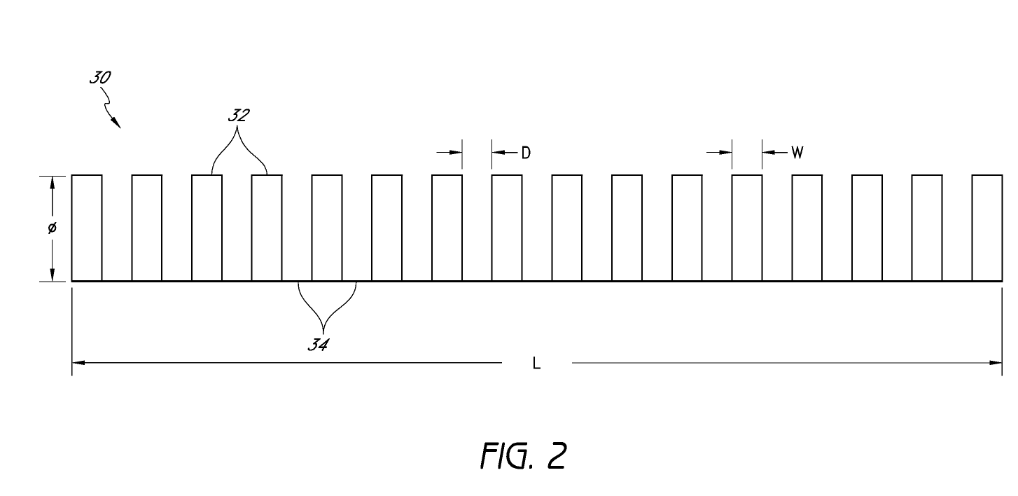

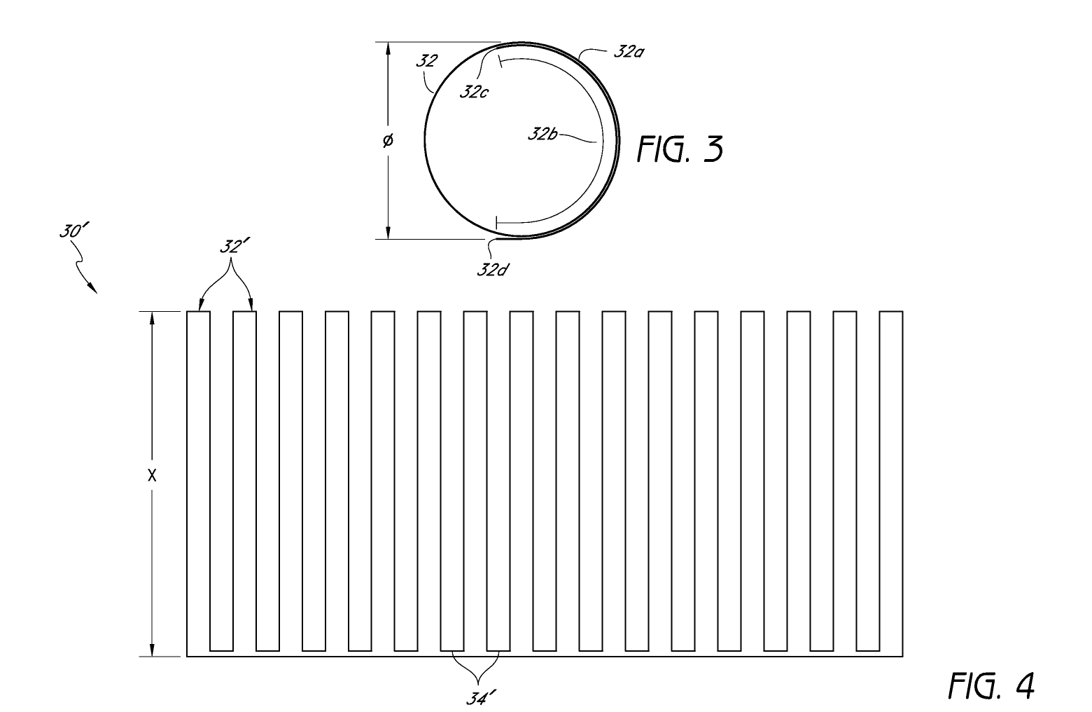

FIG. 1 and FIG. 2 are a perspective view and a side view, respectively, of a stent 30 that can comprise a plurality of annular members 32 interconnected by connection members 34. As illustrated mostly clearly in FIG. 3, which is an enlarged end view of the embodiment of the stent 30 in a pre-placement state, the stent 30 can be configured such that each annular member 32 comprises an exterior portion 32a and an overlapping portion 32b. As used herein, the phrase pre-placement state refers to the configuration of the stent after each annular member of the stent has been formed to the desired annular shape, but before the stent has been positioned within the desired body passage. Thus, in the pre-placement state, no external forces are applied to either the interior or exterior of the stent. As used herein, the phrase post-placement state refers to the state of the stent after the stent has been positioned within the desired body passage. Thus, in the post-placement state, the body passage into which the stent has been positioned generally exerts radial inward forces on one or more of the annular members of the stent.

Each of the annular members 32 can have a spring force bias that, while in the pre-placement state, resists the contraction of each annular member 32. Thus, so that the stent 30 can support and expand the occluded portion of the body passage, the stent 30 can be configured such that, when the stent 30 is in the pre-placement state, one or more of the annular members 32 can be sized such that the outer diameter of such one or more annular member 32 can be greater than the inner diameter of the occluded portion of the body passage.

In some embodiments of the stent 30 or any other stent described herein, one or more of the annular members can be formed so as to have any desired thickness, and so that the thickness of one annular member is generally independent of the thickness of another annular member of the same stent. For example, in some embodiments, the thickness of the annular members positioned near the ends of the stent can be greater than the thickness of the annular members positioned near the middle of the stent. In some embodiments, the thickness of the annular members positioned near the ends of the stent can be less than the thickness of the annular members positioned near the middle of the stent.

The exterior portion 32a can form a complete annulus, while the overlapping portion 32b is defined as the portion of the annular member 32 that can be located adjacent to the inside surface of the exterior portion 32a. In the illustrated embodiment, the overlapping portion 32b forms a semi-circular annular portion through an arc that can be approximately 180 degrees. In some embodiments of any of the annular members disclosed herein, the overlapping portion can exceed 180 degrees. For example, the overlapping portion in any of the annular members can form a complete circle or revolution around the annular member, or more than one complete circle or revolution around the annular member, such as two, three, four, or more complete revolutions around the annular member.

The annular member 32 can be configured such that the circumference of F the annular member 32 remains continuous as the annular member 32 expands, by providing additional material (i.e., the overlapping portion 32b) that can slide relative to the exterior portion 32a of the annular member 32. Stated another way, the annular member 32 can be configured such that the interior end portion 32c of the annular member 32 can be positioned inside of the exterior portion 32a of the annular member 32 and such that the exterior end 32d of the annular member 32 can overlap the interior end portion 32c of the annular member 32 during normal expansion and contract conditions. Thus, as the annular member 32 is expanded, the circumference of the exterior portion 32a increases while the arc length of the overlapping portion 32b decreases.

Maintaining the position of the interior end portion 32c on the interior of the exterior portion 32a can also reduce the instance of end portions of the stent that come into direct contact with body tissue. Further, maintaining the position of the interior end portion 32c on the inside of the exterior portion 32a also can ensure that, as the diameter of the annular member 32 is reduced or contracted, the interior end portion 32c will slide substantially smoothly within the annular member 32 without interference from any body tissue. The interior end portion 32c can be curved to prevent the interior end portion 32c from scratching, gouging, or otherwise damaging the coating on or material comprising the exterior portion 32a as the annular member 32 expands or contracts.

In the illustrated embodiment, the arc length of the overlapping portion 32b can be approximately 50% of the circumference of the exterior portion 32a of the annular member. In some embodiments, the arc length of the overlapping portion 32b can be less than approximately 30%, or approximately 30% to approximately 50%, or approximately 50% to approximately 75%, or approximately 75% to approximately 100%, or more than approximately 100% of the circumference of the exterior portion 32a of the annular member. Any dimensional and geometric configuration described above can be applied to any stent embodiment described or illustrated in this disclosure.

As stated, in the illustrated embodiment, the annular members 32 can be interconnected by connection members 34. The connection members 34 can provide axial support to the stent 30 and secure the individually expandable annular members 32 in the desired orientation and spacing with respect to the other annular members 32. As with any embodiment described in this description, each annular member 32 can be interconnected by one or more connection members 34 located at different radial positions on the annular member 32. In some embodiments, as in the illustrated embodiment, the connection members 34 of the stent 30 can be generally aligned and can interconnect the exterior portion 32a of each of the annular members 32 along the bottom of the stent 30 when the stent 30 is in the orientation depicted in FIGS. 1-3. In some embodiments, the connections members 34 can connect to the overlapping portion 32b instead of the exterior portion 32a of some of the annular members 32. In some embodiments, the connection members 34 of the stent 30 can be misaligned or staggered, such as but not limited to any other arrangements described herein.

In the illustrated embodiment, the length “L” of the stent 32 can be approximately 3.875 inches. In some embodiments, the length “L” of the stent 32 can be less than approximately 2.0 inches, or approximately 2.0 inches to approximately 4.0 inches, or approximately 4.0 inches to approximately 6.0 inches, or approximately 6.0 inches to approximately 8.0 inches, approximately 8.0 inches to approximately 10.0 inches, or approximately 10.0 inches to approximately 12.0 inches, or approximately 12.0 inches to approximately 14.0 inches, or more than approximately 14.0 inches. Any dimensional and geometric configuration described above can be applied to any stent embodiment described or illustrated in this disclosure.

In the illustrated embodiment, the distance “D” between one or more pairs of adjacent annular members 32 can be approximately 0.125 inch. In some embodiments, the distance “D” between annular members 32 can be less than approximately 0.125 inch, or approximately 0.125 inch to approximately 0.250 inch, or approximately 0.250 inch to approximately 0.375 inch, or approximately 0.375 inch to approximately 0.500 inch, or more than approximately 0.500 inch. In some embodiments, the distance “D” between a pair of adjacent annular members 32 can be different than the distance “D” between another pair of adjacent annular members 32. Any dimensional and geometric configuration described above can be applied to any stent embodiment described or illustrated in this disclosure.

In the illustrated embodiment, the width “W” of one or more annular members 32 can be approximately 0.125 inch. In some embodiments, the width “W” of one or more annular members 32 can be less than approximately 0.125 inch, or approximately 0.125 inch to approximately 0.250 inch, or approximately 0.250 inch to approximately 0.375 inch, or approximately 0.375 inch to approximately 0.500 inch, or more than approximately 0.500 inch. In some embodiments, the width “W” of one annular member 32 can be different than the width “W” of another annular member 32 of the same stent 30. Any dimensional and geometric configuration described above can be applied to any stent embodiment described or illustrated in this disclosure.

In the illustrated embodiment, the diameter “Ø” of one or more annular members 32 can be approximately 0.394 inch. In some embodiments, the diameter “Ø” of one or more annular members 32 can be less than approximately 0.125 inch, or approximately 0.125 inch to approximately 0.250 inch, or approximately 0.250 inch to approximately 0.375 inch, or approximately 0.375 inch to approximately 0.500 inch, or approximately 0.500 inch to approximately 0.750 inch, or more than approximately 0.750 inch. In some embodiments, the diameter “Ø” of one annular member 32 can be different than the diameter “Ø” of another annular member 32 of the same stent 30. Any dimensional and geometric configuration described above can be applied to any stent embodiment described or illustrated in this disclosure.

FIG. 4 is a top view of partially formed stent 30′. In this state, the material comprising the stent 30′ has been formed to the desired pre-formed size and shape, such as by, but not limited to, cutting the desired size and shape from a flat sheet of material by any suitable method, including, without limitation, extrusion, chemical etching, stamping, laser cutting, water jet cutting, or by any other suitable method or operation. Accordingly, in the illustrated embodiment, the stent 30′ can be formed from a flat sheet of material. However, in modified embodiments, the stent 30′ can be formed from a plurality of sheets or other components that are connected together via welding, adhesives, stitching, etc. to form the stent 30′. A partially formed stent 30′, such as is illustrated in FIG. 4, can be roll formed or otherwise formed or configured to define the approximate shape of the embodiment of the stent 30 illustrated in FIG. 1. In the illustrated embodiment, the length “X” of each partially formed annular member 32′ illustrated in FIG. 4 can be approximately equal to the sum of the circumference of the exterior portion 32a and the arc length of the overlapping portion 32b of the embodiment of the stent 30 illustrated in FIGS. 1-3. Any of the dimensional and/or geometric configurations described above can be applied to any embodiment described or illustrated in this disclosure.

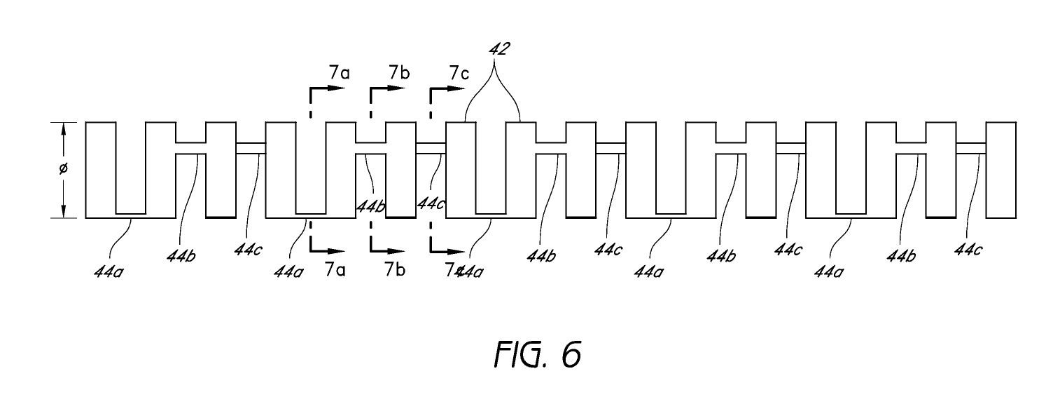

FIGS. 5-8 illustrate a stent 40 comprising a plurality of annular members 42 that can be interconnected by connection members 44a-c. The embodiment illustrated in FIGS. 5-8 can be similar to the embodiments described above, except that the connection members 44a-c are not generally aligned as in the embodiment illustrated in FIGS. 1-4. Rather, the connection members 44a-c can be staggered such that they are located at different points on the annular members. However, the locations of the connection members on each of the annular members for any stent is not limited to the embodiments illustrated and described herein. The connection members can be located at any desired or suitable location on each of the annular members.

In the final formed shape, the connection members 44 can be curved so as to generally match the shape of the formed annular members 42. In some embodiments, where the annular members 42 are generally circular, the connection members 44 can be arcuately curved so as match the generally circular shape of the annular members 42.

Further, for the embodiments illustrated in FIGS. 5-8, the stent 40 can be configured such that the distance “D” between one or more pairs of adjacent annular members 42, the arc length of the overlapping portion 42b of the annular member 42 relative to the circumference of the exterior portion 42a of the annular member 42, the length “L” of the stent 42, the width “W” of one or more annular members 42, and the diameter “Ø” of one or more annular members 42 can be the same as in any of the embodiments described above.

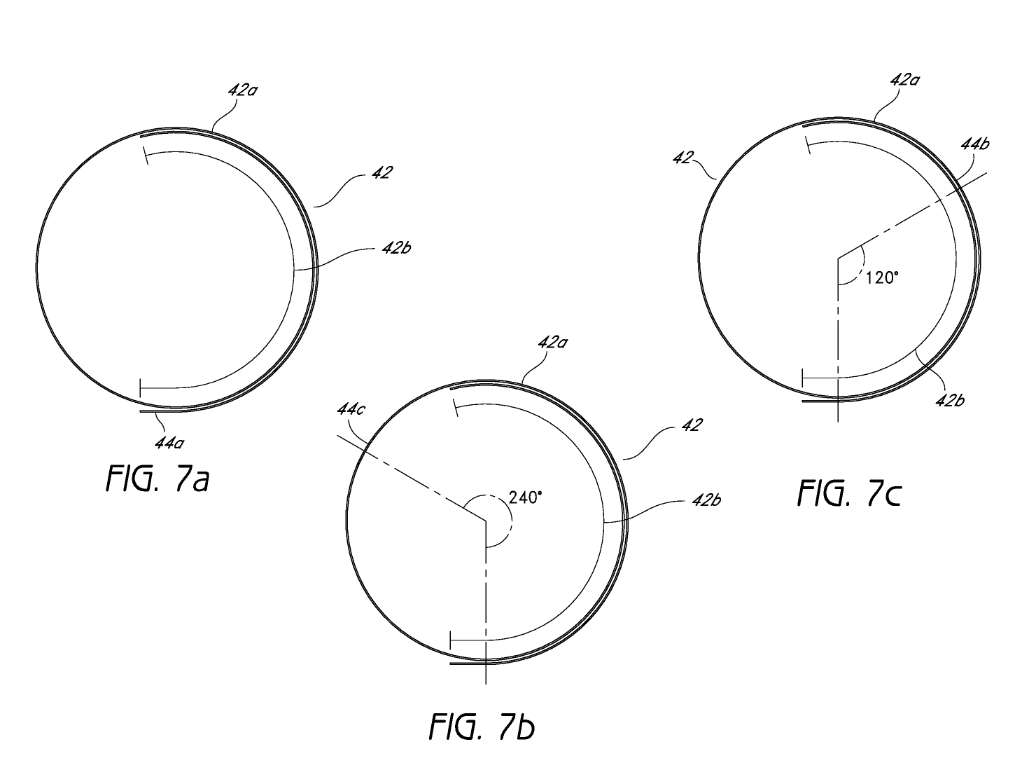

As is most clearly illustrated in FIGS. 7a-7c, each connection segment 44 can be staggered by approximately 120 degrees relative to the adjacent connection segment 44 in either direction. In particular, with reference to FIG. 7a, the connection segment 44a can be located at the bottom of the stent 40 when the stent 40 is in the orientation depicted in FIGS. 5-7. With reference to FIG. 7b, the connection segment 44b can be located at approximately 120 degrees from the bottom of the stent 40 in the counter-clockwise direction. Accordingly, with reference to FIG. 7c, the connection segment 44b can be located at approximately 240 degrees from the bottom of the stent 40, in the counter-clockwise direction.

Thus, in the embodiments illustrated in FIGS. 5-7, each of the connection members 44 can be staggered by approximately 120 degrees relative to the adjacent connection segment 44. Further, each of the connection members 44 can interconnect each of the annular members 32 at the exterior portion 42a of each of the annular members 42. In some embodiments, the connections members 44 can connect to the overlapping portion 42b of some of the annular members 42, instead of the exterior portion 42a. In other embodiments, each of the connection members 44 can be staggered by less than approximately 45 degrees, or approximately 45 degrees to approximately 90 degrees, or approximately 90 degrees to approximately 135 degrees, or approximately 135 degrees to approximately 180 degrees, relative to the adjacent connection segment 44. Additionally, as stated above, each of the annular members 42 can be interconnected by more than one connection member 44, and the radial position of each of the connection members 44 can be staggered.

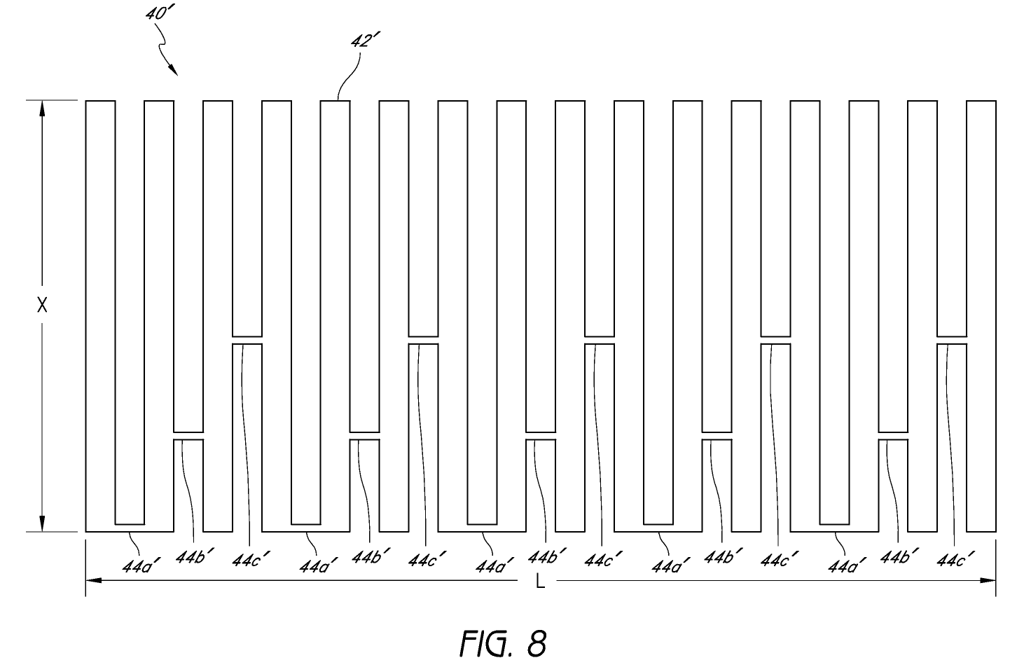

FIG. 8 is a top view of partially formed stent 40′. In this state, the material comprising the stent 40′ has been formed to the desired pre-formed size and shape, such as by, but not limited to, cutting the desired size and shape from a flat sheet of material by any suitable method, including, without limitation, extrusion, chemical etching, stamping, laser cutting, water jet cutting, or by any other suitable method or operation. A partially formed stent 40′, such as is illustrated in FIG. 8, can be roll formed or otherwise formed or configured to define the approximate shape of the embodiment of the stent 40 illustrated in FIG. 5. In the illustrated embodiment, the length “X” of each partially formed annular member 42′ illustrated in FIG. 8 can be approximately equal to the sum of the circumference of the exterior portion 42a and the arc length of the overlapping portion 42b of the embodiment of the stent 40 illustrated in FIGS. 5-7. Any of the dimensional and/or geometric configurations described above can be applied to any embodiment described or illustrated in this disclosure.

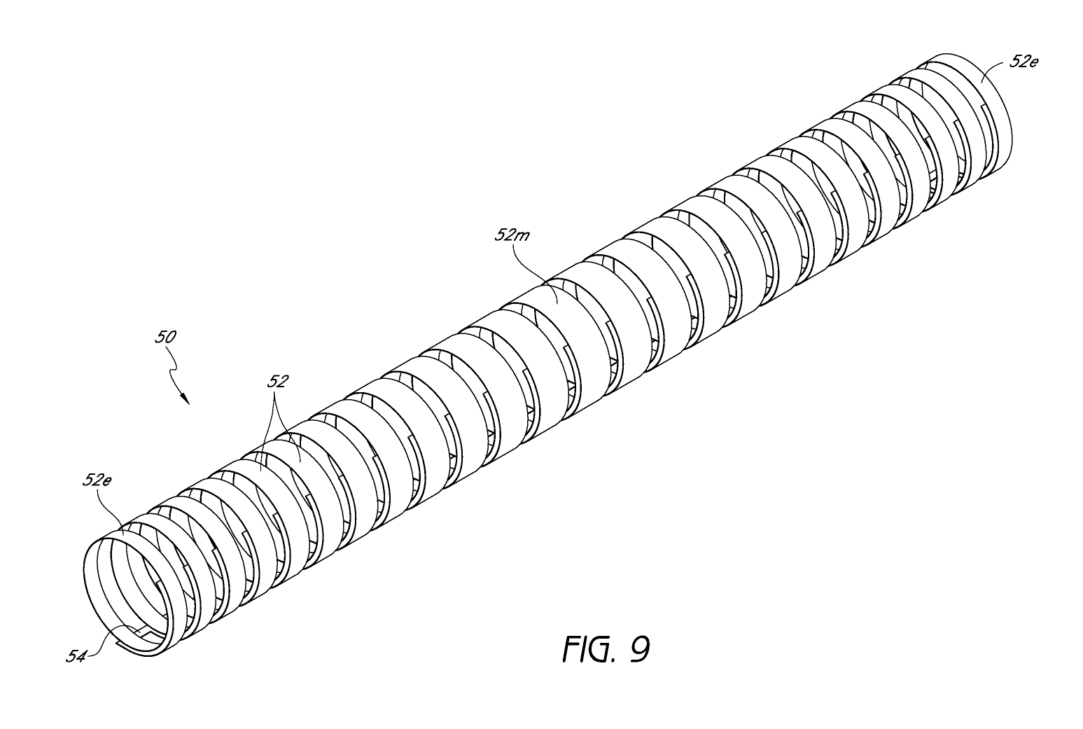

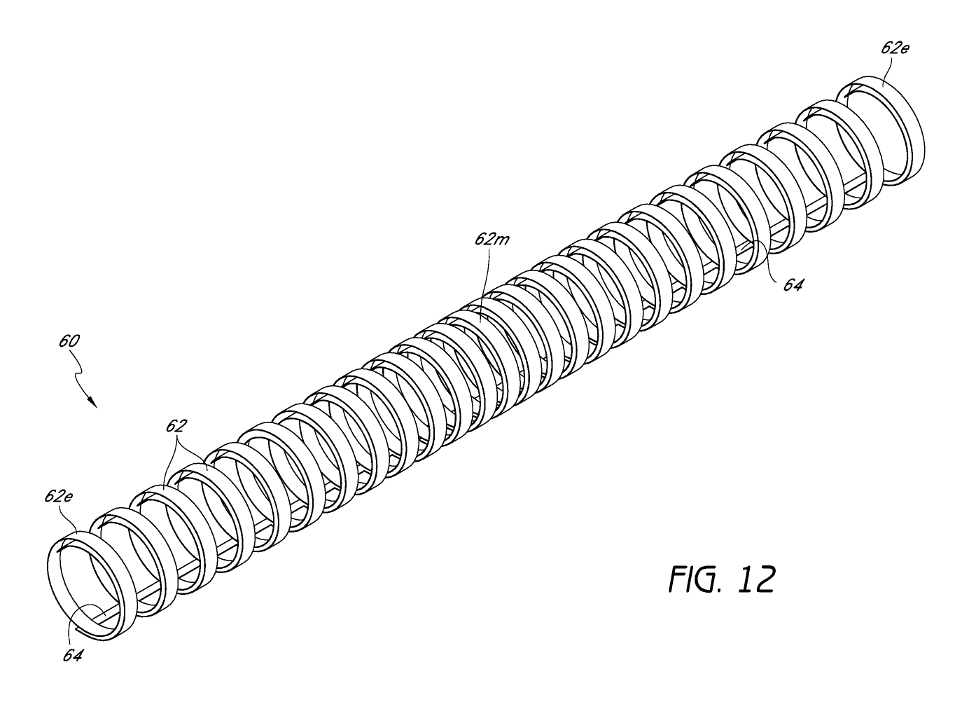

FIGS. 9, 10, and 11 are a perspective view, a side view, and a section view respectively, of another embodiment of a stent 50. In some embodiments, the stent 50 can be configured such that the distance “D” between one or more pairs of adjacent annular members 52, the arc length of the overlapping portion 52b of the annular member 52 relative to the circumference of the exterior portion 52a of the annular member 52, the length “L” of the stent 52, the width “W” of one or more annular members 52, and the diameter “Ø” of one or more annular members 52 can be the same as in any of the embodiments described above. However, the stent 50 can be configured such that the width “W” of each annular member 52 varies. In some embodiments, the stent 50 can be configured such that the width “W” at the centermost annular member 52 can be the widest, the width “W” of each of the two endmost annular members 52 can be the narrowest, and the width “W” of each annular member 52 between the centermost and endmost annular members 52 varies incrementally or non-incrementally from one annular member 52 to the next annular member 52.

In the illustrated embodiment, the distance “D” between each pair of adjacent annular members 52 can be approximately 0.150 inch. In some embodiments, the distance “D” between each pair of annular members 52 can be less than approximately 0.125 inch, or approximately 0.125 inch to approximately 0.250 inch, or approximately 0.250 inch to approximately 0.375 inch, or approximately 0.375 inch to approximately 0.500 inch, or more than approximately 0.500 inch. The connection members 54 can be located at any position described or illustrated with respect to any other embodiments described or illustrated herein, or at any other preferable or suitable position with respect to each annular member 52.

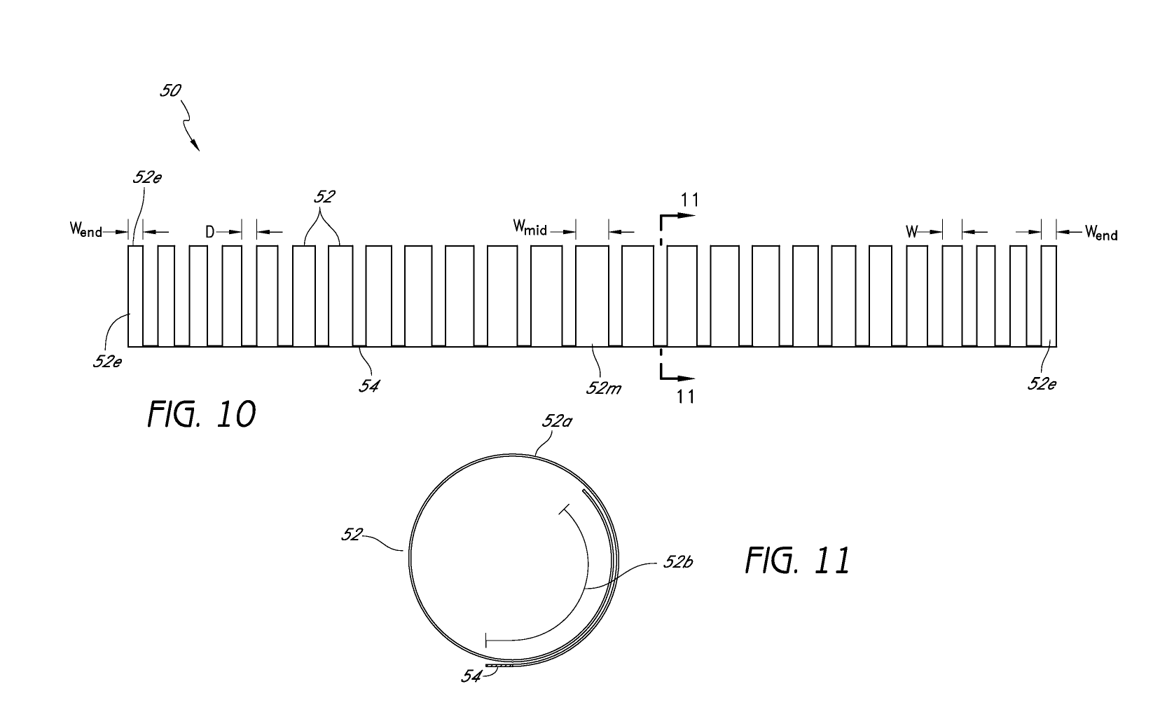

In the illustrated embodiment, the width “W_{mid}” of the annular member 52m located at the approximate center of the stent 50 can be greater than the width “W_{end}” of an annular member 52e located at each approximate end of the stent 50. In particular, in the embodiment illustrated in FIGS. 9-10, the width “W” of each of the twenty-five annular members 52 can incrementally decrease from the width “W_{mid}” to the width “W_{end}”. As illustrated, the width “W_{mid}” of the middle annular member 52m can be approximately 0.330 inch, and the width “W_{end}” of each of the two end annular members 52e can be approximately 0.150 inch. Accordingly, the difference in width between any two adjacent annular members 52 between the annular members 52m and 52e can be approximately 0.015 inch. In some embodiments, the width “W_{mid}” of an annular member 52 located at the approximate center of the stent 50 can be less than width “W_{end}” of an annular member 52 located at the approximate end of the stent 50. Any of the dimensional and/or geometric configurations described above can be applied to any embodiment described or illustrated in this disclosure.

In the illustrated configuration, the annular members 52 located closer to the middle of the stent 50 can be more rigid and, hence, more difficult to contract, than annular members 52 located closer to the end of the stent. Thus, in this configuration, the stent 50 can be positioned in an occluded or diseased body passage such that the mid portion of the stent 50 can be located nearest to the center, or nearest to the most occluded or damaged portion of the body passage, to provide the most expansion and/or support to that region of the body passage. The less rigid, endmost annular members 52 of the stent 50 can be positioned adjacent to portions of the body passage that are less occluded or damaged, and can be more easily contracted and expanded so as to be more flexible within the body passage.

FIGS. 12, 13, and 14 are a perspective view, a side view, and a section view, respectively, of an embodiment of a stent 60. In some embodiments, the stent 60 can be configured such that the distance “D” between one or more pairs of adjacent annular members 62, the arc length of the overlapping portion 62b of the annular member 62 relative to the circumference of the exterior portion 62a of the annular member 62, the length “L” of the stent 62, the width “W” of one or more annular members 62, and the diameter “Ø” of one or more annular members 62 can be the same as in any of the embodiments described above. However, the stent 60 can be configured such that the distance “D” between each annular member 62 varies. Further, as illustrated, in some embodiments, the stent 60 can be configured such that the distance “D_{mid}” between the two centermost annular members 62m can be smaller than the distance “D_{end}” between each of the two endmost annular members 62 and the annular members 62 adjacent thereto, and such that the distance “D” between adjacent annular members 62 incrementally or non-incrementally increases from the middle of the stent 60 to the ends of the stent 60.

In the illustrated embodiment, the width “W” of each annular member 62 can be approximately 0.150 inch. In some embodiments, the width “W” of any annular member 62 of the stent 60 can be less than approximately 0.125 inch, or approximately 0.125 inch to approximately 0.250 inch, or approximately 0.250 inch to approximately 0.375 inch, or approximately 0.375 inch to approximately 0.500 inch, or more than approximately 0.500 inch. The connection members 64 can be located at any position described or illustrated in this disclosure, or at any other preferable or suitable position with respect to each annular member 62.

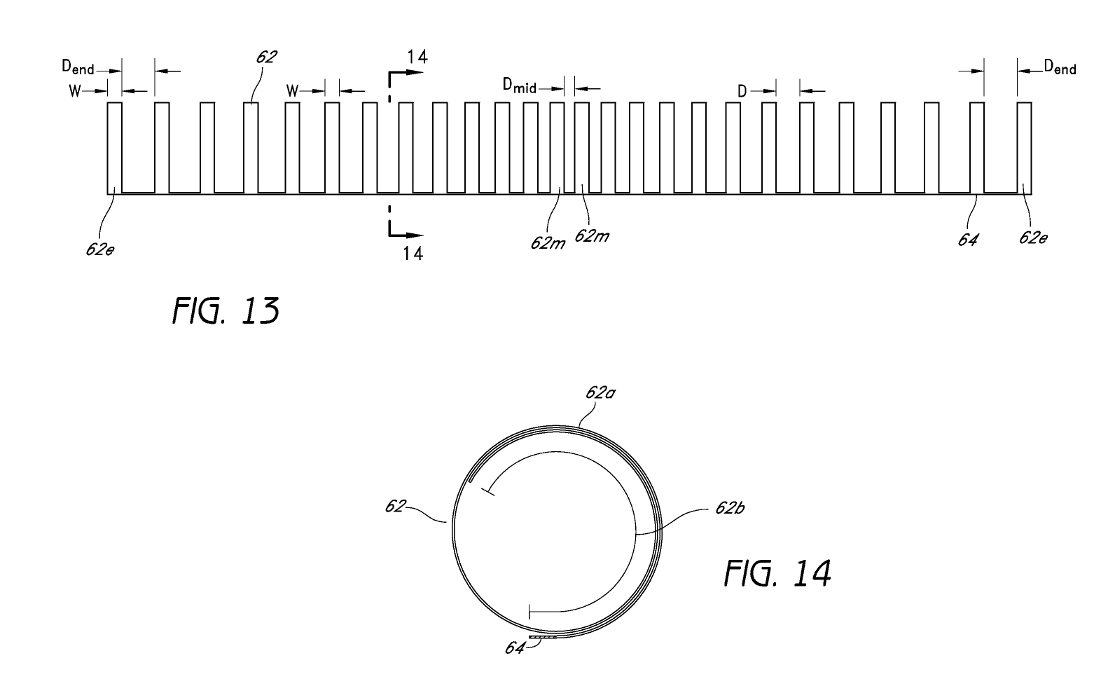

In the illustrated embodiment, the distance “D_{mid}” between the two centermost annular members 62m can be less than the distance “D_{end}” between the endmost annular member 62e and each annular member 62 located adjacent to each endmost annular member 62e. In particular, in the embodiment illustrated in FIGS. 12-13, the distance “D” between each of the twenty-six annular members 62 can incrementally increase from the distance “D_{mid}” to the distance “D_{end}”. As illustrated, the distance “D_{mid}” between the two centermost annular members 62m can be approximately 0.110 inch, and the distance “D_{end}” between the endmost annular member 62e and each annular member 62 located adjacent to each endmost annular member 62e can be approximately 0.350 inch. Accordingly, the difference in distance between any two adjacent pair of annular members 62 can be approximately 0.020 inch. In some embodiments, the distance “D_{mid}” between the two centermost annular members 62m can be greater than the distance “D_{end}” between the endmost annular member 62e and each annular member 62 located adjacent to each endmost annular member 62e. Any of the dimensional and/or geometric configurations described above can be applied to any embodiment described or illustrated herein.

In the illustrated configuration, the middle portion of the stent 60 can provide more annular support to the occluded or diseased body passage than the end portions of the stent 60, and the stent 60 can be more flexible near the ends of the stent 60 than nearer to the middle of the stent 60. In this configuration, the stent 60 can be positioned in the body passage such that the centermost portion of the stent 60, which provides the greatest amount of support and rigidity, will be placed adjacent to the most occluded or damaged portion of the body passage. Consequently, the ends of the stent 60, which provide the least amount of support and rigidity and can be the most flexible, can be positioned adjacent to portions of the body passage that are less occluded or diseased.

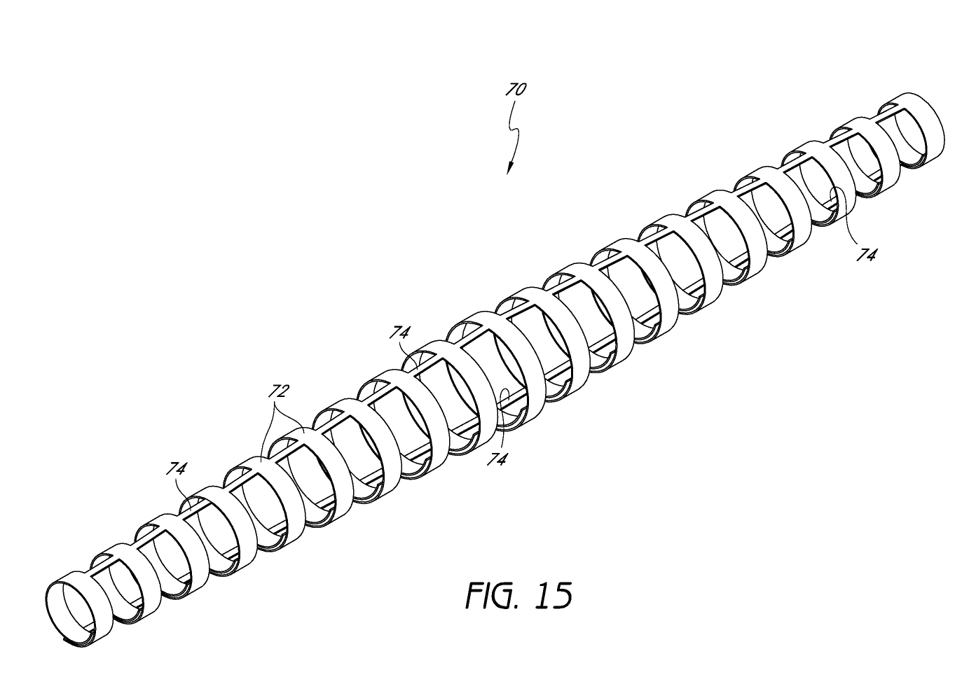

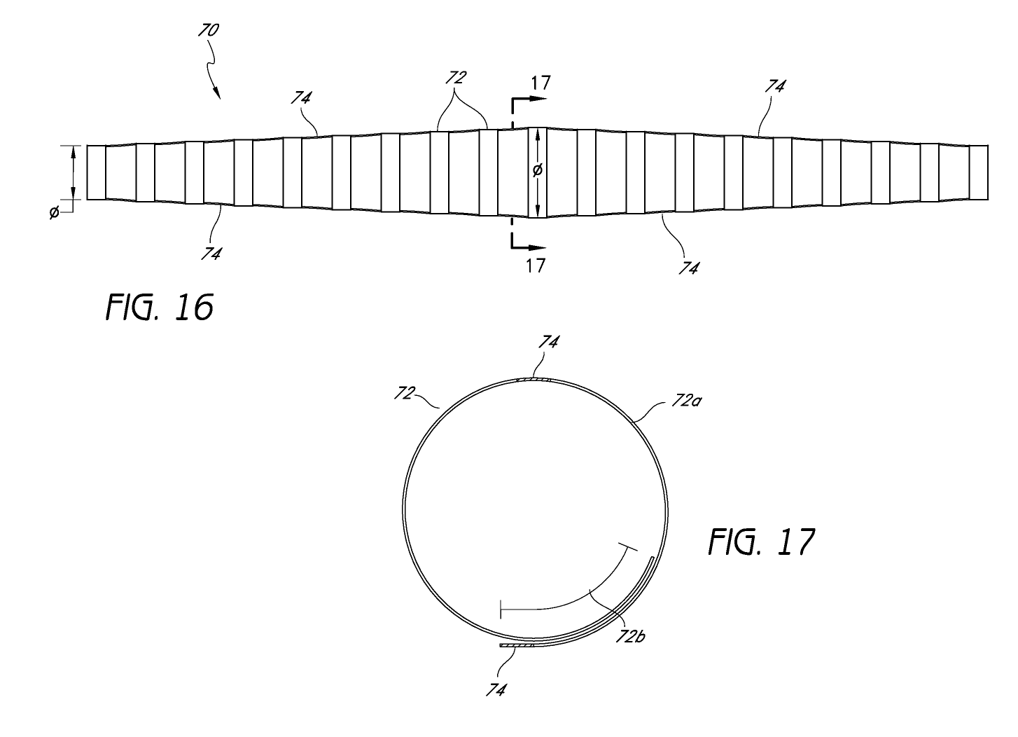

FIGS. 15, 16, and 17 are a perspective view, a side view, and a section view, respectively, of an embodiment of a stent 70. In some embodiments, the stent 70 can be configured such that the distance “D” between one or more pairs of adjacent annular members 72, the arc length of the overlapping portion 72b of the annular member 72 relative to the circumference of the exterior portion 72a of the annular member 72, the length “L” of the stent 72, the width “W” of one or more annular members 72, and the diameter “Ø” of one or more annular members 72 can be the same as in any of the embodiments described above. However, the stent 70 can be configured such that the diameter “Ø” of each annular member 72 can vary from one annular member 72 to the next. In some embodiments, the stent 70 can be configured such that the diameter “Ø” at the centermost annular member 72 can be the largest, the diameter “Ø” of each of the two endmost annular members 72 can be the smallest, and the diameter “Ø” of each annular member 72 between the centermost and endmost annular members 72 varies incrementally or non-incrementally from one annular member 72 to the next annular member 72.

The connection members 74 can be located at any position described or illustrated with respect to any other embodiments described or illustrated herein, or at any other preferable or suitable position with respect to each annular member 72. Furthermore, as with any embodiment described herein, one or more of the annular members 72 can be interconnected by one or more than one connection members 74. In the illustrated embodiment, all of the connection members 74 of the stent 70 can be positioned along the top and bottom of the stent 70 when the stent 70 is in the orientation depicted in FIG. 15, and can interconnect the exterior portion 72a of each of the annular members 72. However, as described above, the connection members 74 can be located at any position described or illustrated with respect to any other embodiments described or illustrated herein, or at any other preferable or suitable position with respect to each annular member 72.

In the illustrated embodiment, the diameter “Ø” of each of the twenty annular members 72 can decrease from the diameter “Ø” of approximately 0.500 inch at the centermost portion of the stent 70, to a diameter “Ø” of approximately 0.300 inch at either endmost portion of the stent 70. Any of the dimensional and/or geometric configurations described above can be applied to any embodiment described or illustrated with respect to any other embodiments described or illustrated herein.

In the illustrated configuration, the annular members 72 located closer to the middle of the stent 70 can expand more than the annular members 72 located closer to the end of the stent 70. Thus, in this configuration, the stent 70 can be positioned in an occluded or diseased body passage such that the mid portion of the stent 70 can be located nearest to the center, or nearest to the most occluded or damaged portion of the body passage, to provide the most expansion and/or support to that region of the body passage. The less expansive endmost annular members 72 of the stent 70 can be positioned adjacent to portions of the body passage that are less occluded or damaged, and can exert a smaller expansive force on the inside walls of the body passage as compared to the annular members 72 positioned nearer to the middle of the stent 70. The dimensional and geometric configurations described herein can be applied to any embodiment described or illustrated in this disclosure.

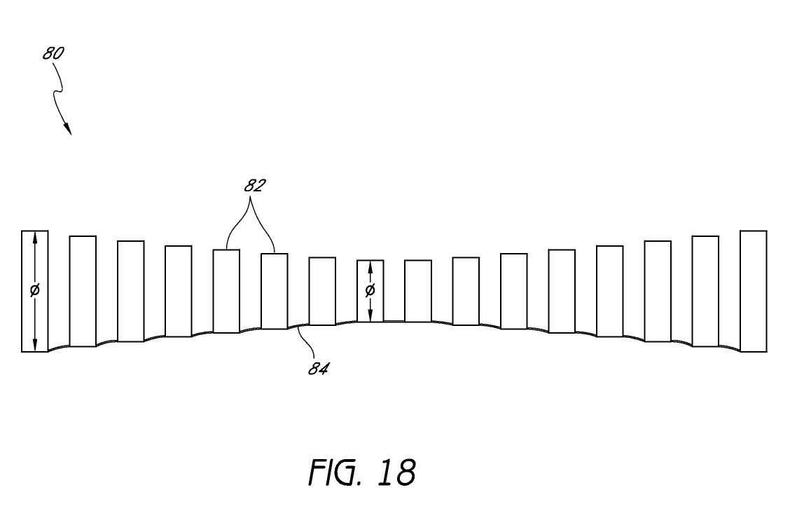

As illustrated in FIG. 18, in some embodiments, the diameter “Ø” of an annular member 82 located at the approximate center of the stent 80 can be less than diameter “Ø” of an annular member 82 located at the either of the approximate ends of the stent 80. The dimensional and geometric configurations described herein can be applied to any embodiment described or illustrated in this disclosure.

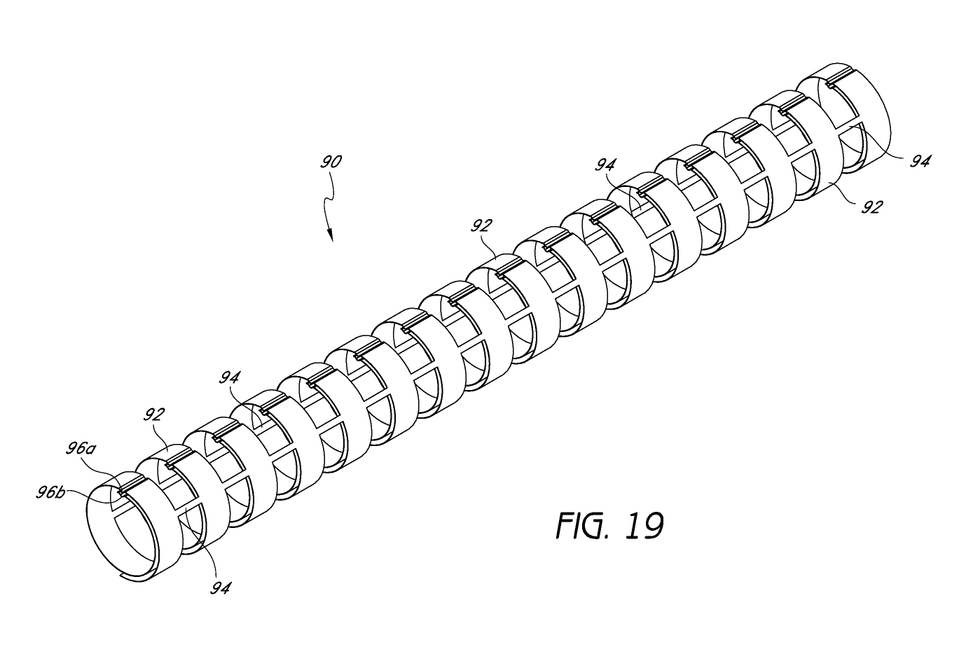

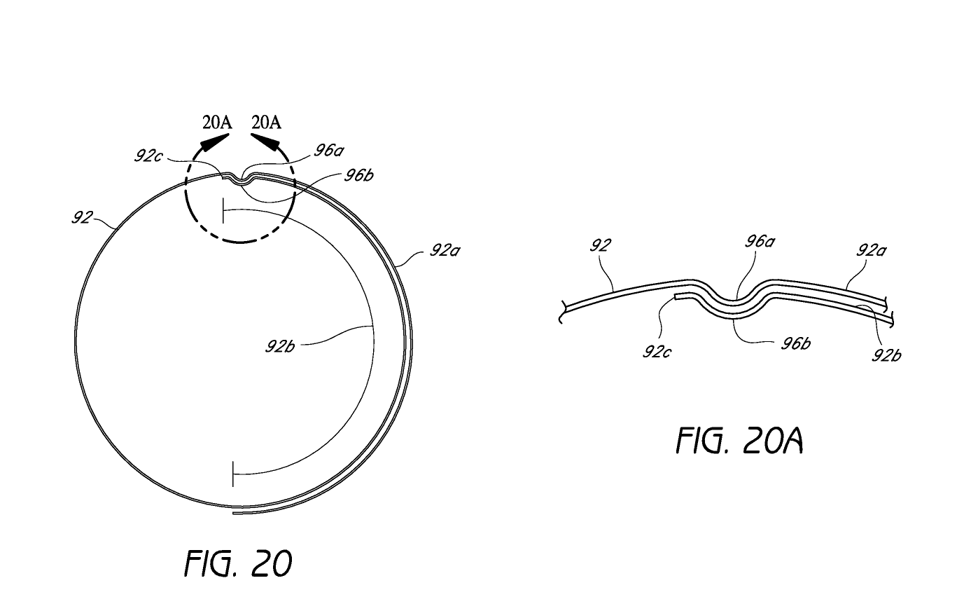

FIGS. 19, 20, and 20A are a perspective view, an enlarged end view, and a further enlarged view of an end portion, respectively, of an embodiment of a stent 90. In some embodiments, the stent 90 can be configured such that the distance “D” between one or more pairs of adjacent annular members 92, the arc length of the overlapping portion 92b of the annular member 92 relative to the circumference of the exterior portion 92a of the annular member 92, the length “L” of the stent 92, the width “W” of one or more annular members 92, and the diameter “Ø” of one or more annular members 92 can be the same as in any of the embodiments described above.

Additionally, one or more of the annular members 92 in the illustrated embodiment can comprise an outer groove 96a formed in the exterior portion 92a of the annular member 92, and an inner groove 96b formed in the overlapping portion 92b of the annular member 92. In some embodiments, the grooves 96a, 96b provide a relative bias that releasably secures the annular member 92 at a particular diameter defined by the locations of each of the grooves 96a, 96b. In the illustrated embodiment, the bias that results from the grooves 96a, 96b can be overcome by exerting a force on the annular member 92 sufficient to move the grooves 96a, 96b relative to one another such that the grooves 96a, 96b are no longer aligned. The size and geometric configuration of the grooves 96a, 96b can control the amount of force required to overcome the bias from the grooves 96a, 96b. In some embodiments, increasing the depth of the grooves 96a, 96b will increase the amount of force required to overcome the bias from the grooves 96a, 96b, while decreasing the depth of the grooves 96a, 96b will decrease the amount of force required to overcome the bias from the grooves 96a, 96b.

The connection members 94 can be located at any position described or illustrated with respect to any other embodiments described or illustrated in this disclosure, or at any other preferable or suitable position with respect to each annular member 92. Furthermore, as with any embodiment described herein, one or more of the annular members 92 can be interconnected by one or more than one connection members 94. In the illustrated embodiment, all of the connection members 94 of the stent 90 can be positioned each of the laterals sides of the stent 90 when the stent 90 is in the orientation depicted in FIG. 15, and can interconnect the exterior portion 92a of each of the annular members 92. However, as described above, the connection members 94 can be located at any position described or illustrated in this disclosure, or at any other preferable or suitable position with respect to each annular member 92.

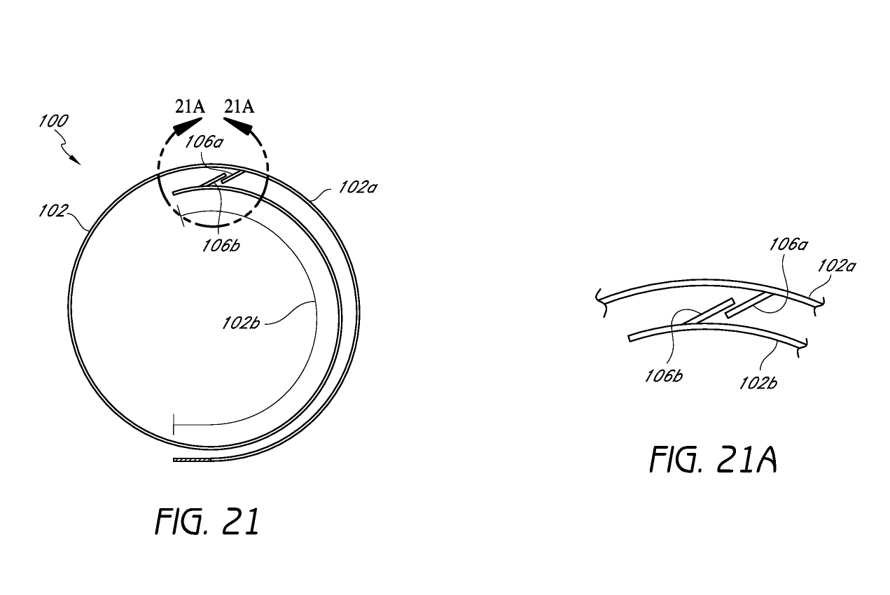

In some embodiments, as illustrated in FIGS. 21 and 21A, the tabs 106a, 106b located on the annular member 102 of stent 100 can be configured to provide a locking bias that substantially, non-releasably secure the annular member 102 at a particular diameter defined by the locations of each of the tabs 106a, 106b. In the illustrated configuration, the tabs 106a, 106b can be configured so as to not restrict the further expansion of the annular members 102. However, in the illustrated configuration, the tabs 106a, 106b can be configured so as to not restrict the annular members 102 from constricting beyond a preferred minimum diameter, based on the location of the tabs 106a, 106b relative to one another. In other embodiments (not illustrated), the tabs 106a, 106b can be configured to restrict the expansion of the annular members 102 while not restricting the contraction of the annular members.

In some embodiments, the stent 100 can be configured such that the distance “D” between one or more pairs of adjacent annular members 102, the arc length of the overlapping portion 102b of the annular member 102 relative to the circumference of the exterior portion 102a of the annular member 102, the length “L” of the stent 102, the width “W” of one or more annular members 102, and the diameter “Ø” of one or more annular members 102 can be the same as in any of the embodiments described above.

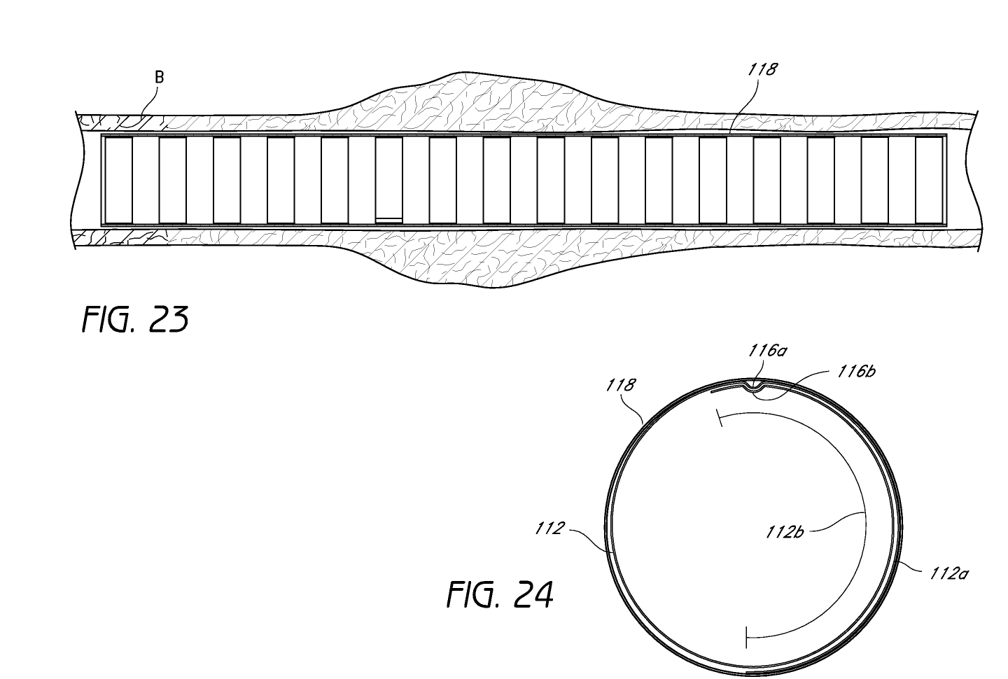

FIG. 22 is a partially sectioned perspective view of another embodiment of a stent 110. FIG. 23 is a section view of the embodiment of the stent 110 shown in FIG. 22, positioned within a body passage. FIG. 24 is an enlarged end view of the embodiment of the stent 110 shown in FIG. 22 (body passage not illustrated). As illustrated in FIGS. 22, 23, and 24, the stent 110 can comprise a sheath 118 (also referred to herein as a sleeve or graft). In some embodiments, the sheath 118 can help maintain the individually expandable annular members 112 in the desired orientation and spacing relative to other annular members 112 and/or body vessel. The sheath 118 can be configured to be a thin, hollow, flexible cylindrical member that can be formed from any suitable material, such as, without limitation, a non-reactive polymeric material. In some embodiments, the sheath 118 can be formed from PTFE or ePTFE.

In some embodiments, where the sheath 118 is configured to provide axial support to the annular members 112, as in the illustrated embodiment, the stent 110 can be configured so as to not have any connection members interconnecting the annular members 112. In some embodiments, the stent 110 can comprise connection members configured to interconnect the annular members, at any of the positions or having any of the sizes and/or geometries described above.

Adhesive material or any other suitable other bonding agent can be used to adhere the annular members 112 to the sheath 118. In some embodiments, the annular members 112 can be mechanically held in the desired location with respect to the sheath 118. In some embodiments, the annular members 112 can be supported by or attached to the sheath 118 using sutures. In some embodiments, the annular members 112 can be attached to the sheath 118 by passing sutures through one or more holes created in one or more of the annular members 112. The suture holes can be formed during the forming process used to create the size and shape of the annular members 112 or other features of the stent 110.

In some embodiments, the stent 110 can be configured such that the distance “D” between one or more pairs of adjacent annular members 112, the arc length of the overlapping portion 112b of the annular member 112 relative to the circumference of the exterior portion 112a of the annular member 112, the length “L” of the stent 112, the width “W” of one or more annular members 112, and the diameter “Ø” of one or more annular members 112 can be the same as in any of the embodiments described above.

Additionally, the illustrated embodiment can comprise an outer groove 116a formed in the exterior portion 112a of the annular member 112, and an inner groove 116b formed in the overlapping portion 112b of the annular member 112. In some embodiments, the grooves 116a, 116b provide a relative bias that releasably secures the annular member 112 at a particular diameter defined by the locations of each of the grooves 116a, 116b. In the illustrated embodiment, the bias that results from the grooves 116a, 116b can be overcome by exerting a force on the annular member 112 sufficient to move the grooves 116a, 116b relative to one another such that they are not aligned. The size and geometric configuration of the grooves 116a, 116b can control the amount of force required to overcome the bias from the grooves 116a, 116b. In some embodiments, increasing the depth of the grooves 116a, 116b will increase the amount of force required to overcome the bias from the grooves 116a, 116b, while decreasing the depth of the grooves 116a, 116b will decrease the amount of force required to overcome the bias from the grooves 116a, 116b. In some embodiments, the annular members 112 can be formed without grooves 116a, 116b and can be similar to the individual annular members of any of the stents described above.

The stent 110 illustrated in FIG. 23 can be percutaneously positioned and delivered via a delivery catheter in the desired body passage B with the sheath 118 surrounding and supporting each of the annular members 112. In the illustrated embodiment, the sheath 118 can be terminated at the outermost edge of the two outermost annular members 112. In some embodiments, the sheath 118 can be terminated within the outermost edge of the two outermost annular members 112. In some embodiments, the sheath 118 can be terminated outside of the outermost edge of the two outermost annular members 112.

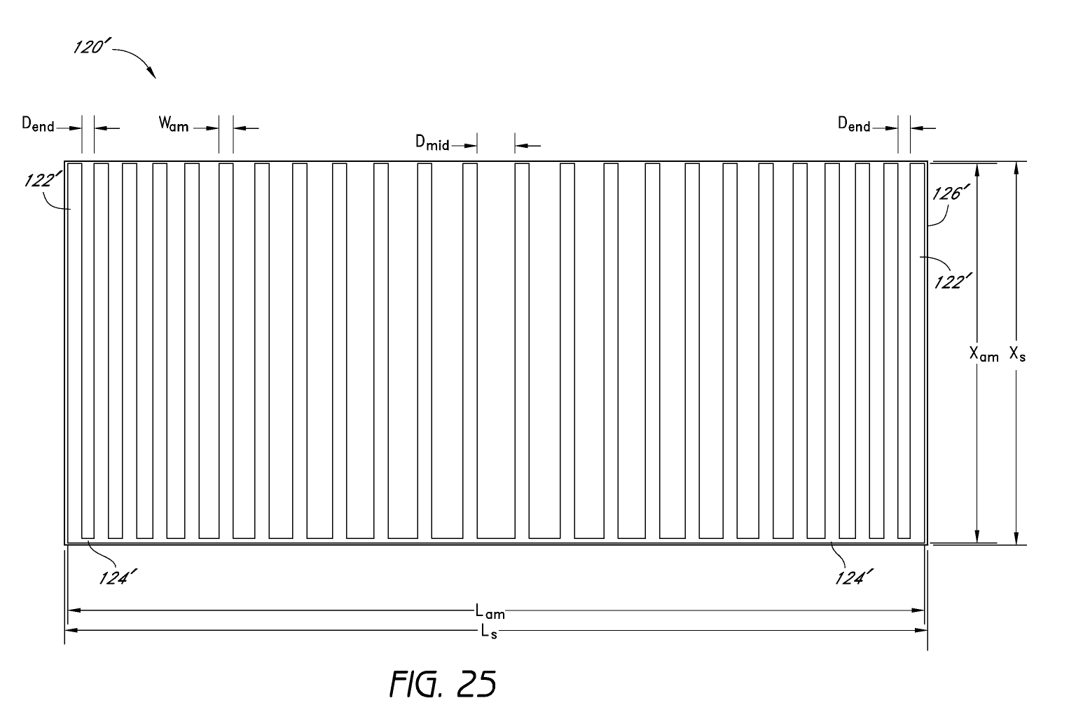

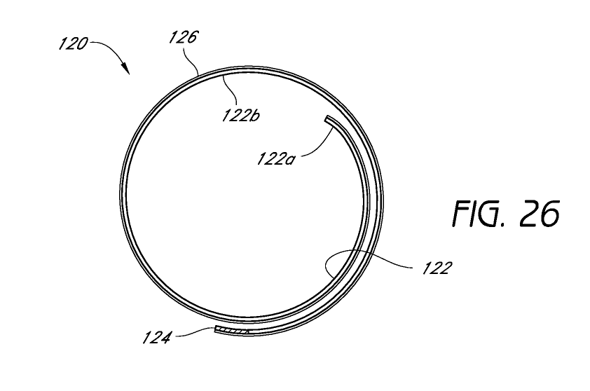

FIG. 25 is a top view of another embodiment of a stent 120′, showing the stent 120′ in a partially formed state. FIG. 26 is an enlarged end view of the embodiment of the stent 120 shown in FIG. 25, showing the stent in a rolled or formed state. In the partially formed state, as shown in FIG. 25, the material comprising the stent 120′ has been formed to the desired pre-formed size and shape, such as by, without limitation, cutting the desired size and shape from a flat sheet of material by any suitable method, including, without limitation, extrusion, chemical etching, stamping, laser cutting, water jet cutting, or by any other suitable method or operation. Additionally, in some embodiments, any of the features, dimensional and/or geometric configurations, or other details described or illustrated in this disclosure or known in the art can be applied to the stent 120.

In some embodiments, as in the illustrated embodiment, the stent 120′, 120 can be sized and shaped so that the width of the space between the annular members 122′, 122 at the midpoint of the stent (represented by “D_{mid}” in FIG. 25) is greater than the width of the space between the annular members 122′, 122 near the edges of the stent (represented by “D_{end}” in FIG. 25). In some embodiments, this configuration of the stent 120′, 120 can permit the stent 120 to be more flexible in the mid region, while permitting the stent 120 to provide greater radial support at the outer regions. In some embodiments (not illustrated), the stent 120′, 120 can be sized and shaped so that the width D_{mid} of the space between the annular members 122′, 122 at the midpoint of the stent is less than the width D_{end} of the space between the annular members 122′, 122 near the edges of the stent. In some embodiments, the width of the annular members 122′, 122 (represented by “Wₐₘ” in FIG. 25) can be varied so that one or more of the annular members 122′, 122 defines a width that is greater or less than the width of other annular members 122′, 122.

In some embodiments, as illustrated in FIG. 26, the stent 120 can be formed so that the interior end portion 122a of one or more of the annular members 122 is curved inwardly. In this configuration, the inwardly curved interior end portion 122a can improve the ability of the interior end portion 122a to slide smoothly relative to the inside surface 122b of the one or more of the annular members 122 and can potentially improve the dynamic responsiveness of the annular members 122.

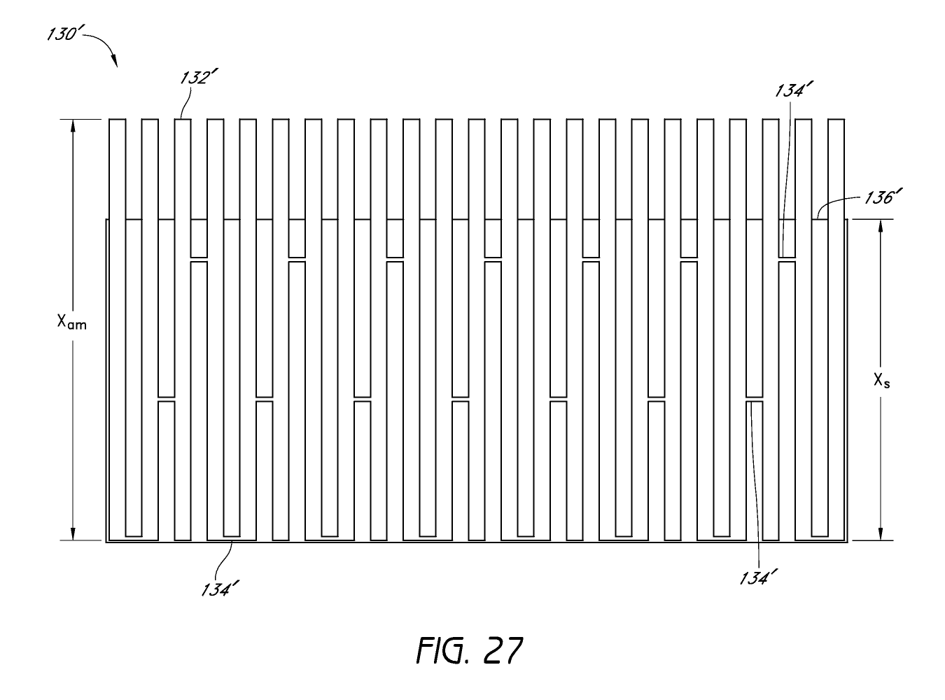

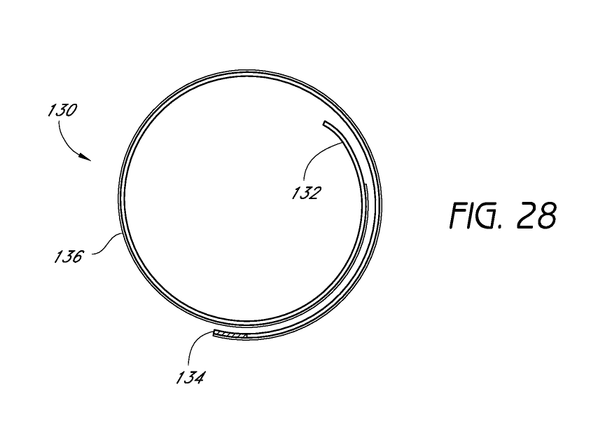

In some embodiments, the stent 120′, 120 can be formed so as to have connection members 124′, 124 that interconnect each of the annular members 122′, 122. In some embodiments, the connection members 124′, 124 can be located as illustrated in FIGS. 25-26. In some embodiments, the connection members 124′, 124 can be located in the same or similar positions as with the connection members 134′, 134 of the stent 130′, 130 illustrated in FIGS. 27-28 (which are a top view of another embodiment of a stent 130′ in a partially formed state and an enlarged side view of the embodiment of the stent 130′ shown in FIG. 27, showing the stent 130 in a formed state, respectively). In some embodiments, the connection members 124′, 124 can be located at any position described or illustrated with respect to any other embodiments described or illustrated in this disclosure, or at any other preferable or suitable position with respect to each annular member 122′, 122. Furthermore, as with any embodiment described herein, one or more of the annular members 122′, 122 can be interconnected by one or more than one connection member 124′, 124. In the illustrated embodiment, all of the connection members 124′, 124 of the stent 120′, 120 can be positioned along the bottom edge of the preformed stent 120′ when the stent 120′ is in the orientation depicted in FIG. 25. However, as described above, the connection members 124′, 124 can be located at any position described or illustrated with respect to any other embodiments described or illustrated herein.

In some embodiments, in the final formed shape as illustrated in FIG. 26, the connection members 124′, 124 can be curved so as to generally match the shape of the formed annular members 122′, 122. In some embodiments, where the annular members 122′, 122 are generally circular, the connection members 124′, 124 can be arcuately curved so as match the generally circular shape of the annular members 122′, 122.

Additionally, as illustrated in FIGS. 25-26, the stent 120′, 120 can comprise a sheath 126′, 126 which, among other functions, can help maintain the individually expandable annular members 122′, 122 in the desired orientation and spacing relative to other annular members 122′, 122 and/or body vessel. In the pre-formed state shown in FIG. 25, the sheath 126′ can be formed from a thin, flat sheet of any suitable material, such as, without limitation, a non-reactive polymeric material. In some embodiments, the sheath 126′, 126 can be formed from PTFE or ePTFE.

With reference to FIG. 25, in some embodiments, as in the illustrated embodiment, the length “Xₐₘ” of each partially formed annular member 122′ can be approximately equal to the length “Xₛ” of the sheath 126′. In this configuration, in the formed state as illustrated in FIG. 26, the sheath 126 can cover approximately the entire length of each annular member 122. In some embodiments, as is illustrated in FIGS. 27-28, the length “Xₐₘ” of some or all of the partially formed annular members 132′ of the stent 130′ can be greater than the length “Xₛ” of the sheath 136′ so that portions of the annular members 122′, 122 are not covered by the sheath.

In some embodiments, as in the illustrated embodiment, the stent 120′, 120 can be sized and/or shaped so that the length of the plurality of annular members 122′, 122 (represented by “Lₐₘ” in FIG. 25) can be approximately equal to the length of the sheath 126′, 126 (represented by “Lₛ” in FIG. 25). However, in some embodiments (not illustrated) of the stent 120′, 120 or any other stent disclosed herein, the stent 120′, 120 can be sized and/or shaped so that the length Lₐₘ of the plurality of annular members 122′, 122 can be generally less than the length Lₛ of the sheath 126′, 126 and the sheath 126′, 126 can be wrapped around an edge of the plurality of annular members 122′, 122.

In some embodiments (not illustrated) of the stent 120′, 120 or any other stent disclosed herein, the stent 120′, 120 can be sized and shaped so that the length Lₐₘ of the plurality of annular members 122′, 122 can be generally greater than the length Lₛ of the sheath 126′, 126 such that the sheath 126′, 126 covers only a portion of the length Lₛ of the sheath 126′, 126. In some embodiments, some or all of the annular members 122′, 122 can be configured to encourage tissue growth into at least a portion of the annular members 122′, 122 so as to help secure at least a portion of the annular members 122′, 122 to the vessel wall. In this configuration, the annular members 122′, 122 that are not covered by the sheath 126′, 126 can be configured to help secure the stent 120′, 120 to the vessel wall.

In some embodiments, where the sheath 126′, 126 can be configured to provide axial support to the annular members 122′, 122, as in the illustrated embodiment, the stent 120′, 120 can be configured so as to not have any connection members interconnecting the annular members 122′, 122. In some embodiments, the stent 120′, 120 can comprise connection members configured to interconnect the annular members 122′, 122, at any of the positions or having any of the sizes and/or geometries described above.

Adhesive material or any other suitable material, bonding agent, or other method can be used to adhere or affix the annular members 122′, 122 to the sheath 126′, 126. In some embodiments, the annular members 122′, 122 can be mechanically held in the desired location with respect to the sheath 126′, 126. In some embodiments, the annular members 122′, 122 can be supported by or attached to the sheath 126′, 126 using sutures. In some embodiments, the annular members 122′, 122 can be attached to the sheath 126′, 126 by passing sutures through one or more holes created in one or more of the annular members 122′, 122. The suture holes can be formed during the forming process used to create the size and shape of the annular members 122′, 122 or other features of the stent 120′, 120.

A partially formed stent 120′, such as is illustrated in FIG. 25, can be roll formed or otherwise formed or configured to define the approximate circular shape of the embodiment of the stent 120, of which an end view is illustrated in FIG. 26. In some embodiments, the stent 120 can be formed by rolling the sheath 126′ together with the annular members 122′. In some embodiments, the sheath 126′ can be secured to the annular members 122′, 122 before or after the annular members are formed into the desired final shape, which can be, but is not required to be, generally annular. In the embodiment of the stent 120′, 120 illustrated in FIGS. 25-26, the sheath 126′, 126 can be positioned on the outside surface of the annular members 122′, 122 so as to be positioned generally between the stent 122 and the body vessel, as illustrated in FIG. 26. In some embodiments (not illustrated), the sheath 126′, 126 can be positioned on the inside surface of the annular members 122′, 122 so that the annular members 122′, 122 are positioned generally between the sheath 126 and the body vessel.

In some embodiments (not illustrated), the annular members 122′, 122 can be positioned between two layers of sheath material or embedded within the sheath material so that the sheath 126 is positioned between the annular members 122′, 122 and the body vessel, and also on the inside surface of the annular members 122′, 122. For example, in some embodiments, the annular members 122′, 122 can be laminated between two sheets of sheath material of the same or different size and/or shape. In some embodiments, the annular members 122′, 122 can be embedded in the sheath material so as to form a single, integral unit. In some embodiments, this can be achieved by melting two sheets of sheath material together after the annular members 122′, 122 have been positioned therebetween. In some embodiments, a single sheet of sheath material can be wrapped around the one or more of the edges of the annular members 122′, 122 and/or connection members.

As discussed above, FIG. 27 is a top view of another embodiment of a stent 130′, showing the stent in a partially formed state. FIG. 28 is an enlarged end view of the embodiment of the stent 130 shown in FIG. 27, showing the stent 130 in a rolled or formed state. In the partially formed state, as shown in FIG. 27, the material comprising the stent 130′ has been formed to the desired pre-formed size and shape, such as by, without limitation, cutting the desired size and shape from a flat sheet of material by any suitable method, including, without limitation, extrusion, chemical etching, stamping, laser cutting, water jet cutting, or by any other suitable method or operation. Additionally, in some embodiments, any of the features, dimensional and/or geometric configurations, or other details described or illustrated in this disclosure or known in the art can be applied to the stent 130.

In some embodiments, the connection members 134′, 134 that can interconnect the annular members 132′, 132 can be located at any desired position, including as illustrated in FIG. 27. In some embodiments, the connection members 134′, 134 can be located at any position described or illustrated with respect to any other embodiments described or illustrated in this disclosure, or at any other preferable or suitable position with respect to each annular member 132′, 132. Furthermore, as with any embodiment described herein, one or more of the annular members 132′, 132 can be interconnected by one or more than one connection member 134′, 134.

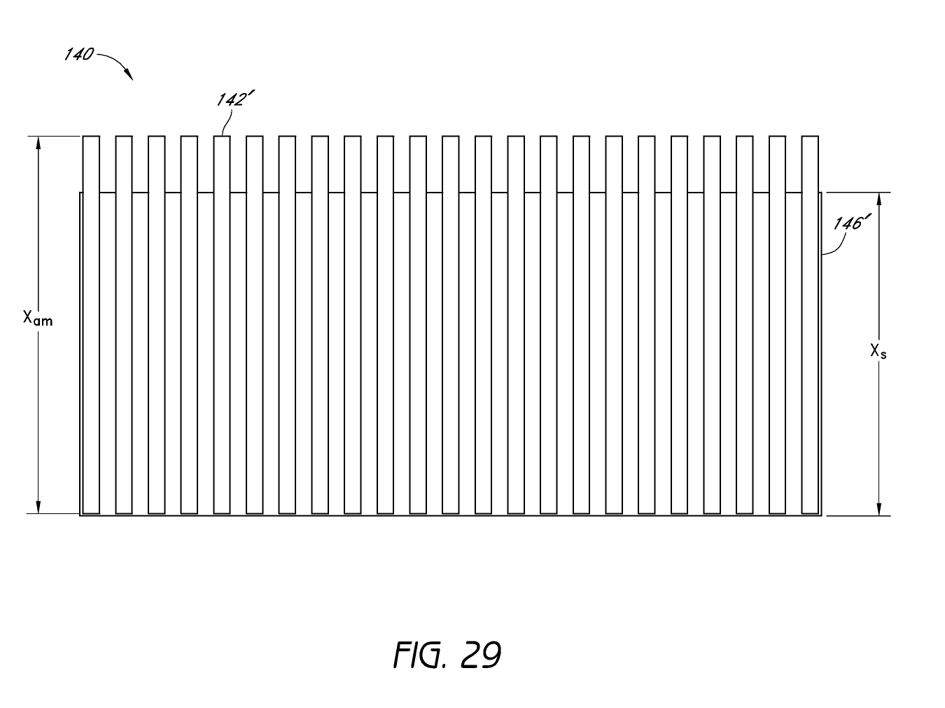

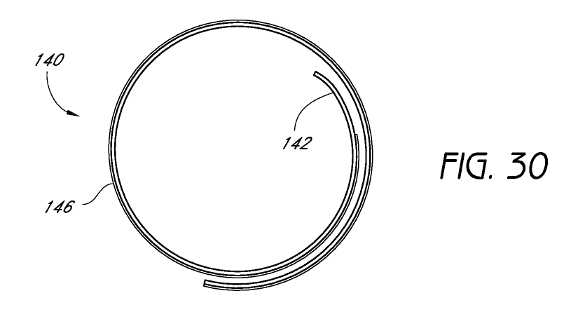

FIG. 29 is a top view of another embodiment of a stent 140′, showing the stent in a partially formed state. FIG. 30 is an enlarged end view of the embodiment of the stent 140 shown in FIG. 29, showing the stent 140 in a rolled or formed state. In the partially formed state, as shown in FIG. 29, the material comprising the stent 140′ has been formed to the desired pre-formed size and shape, such as by, without limitation, cutting the desired size and shape from a flat sheet of material by any suitable method, including, without limitation, extrusion, chemical etching, stamping, laser cutting, water jet cutting, or by any other suitable method or operation. Additionally, in some embodiments, any of the features, dimensional and/or geometric configurations, or other details described or illustrated in this disclosure or known in the art can be applied to the stent 140.

In some embodiments (not illustrated), the stent 140 can be formed so that the interior end portion of one or more of the annular members 142 is curved inwardly. In this configuration, the inwardly curved interior end portion can improve the ability of the interior end portion to slide smoothly relative to the inside surface of the one or more of the annular members 142, and can potentially improve the dynamic responsiveness of the annular members 142. In some embodiments, as in the illustrated embodiment, the stent 140 can be formed so that the interior end portion of one or more of the annular members 142 is curved to match the contour of the surface against which it abuts.

In some embodiments, as in the illustrated embodiment, the stent 140′, 140 can be formed without connection members. Additionally, as illustrated in FIGS. 29-30, the stent 140′, 140 can comprise a sheath 146′, 146 which, among other functions, can help maintain the individually expandable annular members 142′, 142 in the desired orientation and spacing relative to other annular members 142′, 142 and/or body vessel. In the pre-formed state shown in FIG. 29, the sheath 146′ can be formed from a thin, flat sheet of any suitable material, such as, without limitation, a non-reactive polymeric material. In some embodiments, the sheath 146′, 146 can be formed from PTFE or ePTFE.

In some embodiments (not illustrated), the annular members 142′, 142 can be positioned between two layers of sheath material or embedded within the sheath material so that the sheath 146 is positioned between the annular members 142′, 142 and the body vessel, and also on the inside surface of the annular members 142′, 142. For example, in some embodiments, the annular members 142′, 142 can be laminated between two sheets of sheath material. In some embodiments, a single sheet of sheath material can be wrapped around the one or more of the edges of the annular members 142′, 142 and/or connection members.

In some embodiments, as is illustrated in FIGS. 29-30, the length “Xₐₘ” of some or all of the partially formed annular member 142′ of the stent 140′ can be greater than the length “Xₛ” of the sheath 146′ so that portions of the annular members 142′, 142 are not covered by the sheath. In some embodiments, where the sheath 146′, 146 can be configured to provide axial support to the annular members 142′, 142, as in the illustrated embodiment, the stent 140′, 140 can be configured so as to not have any connection members interconnecting the annular members 142′, 142. In some embodiments, the stent 140′, 140 can comprise connection members configured to interconnect the annular members 142′, 142, at any of the positions or having any of the sizes and/or geometries described above.

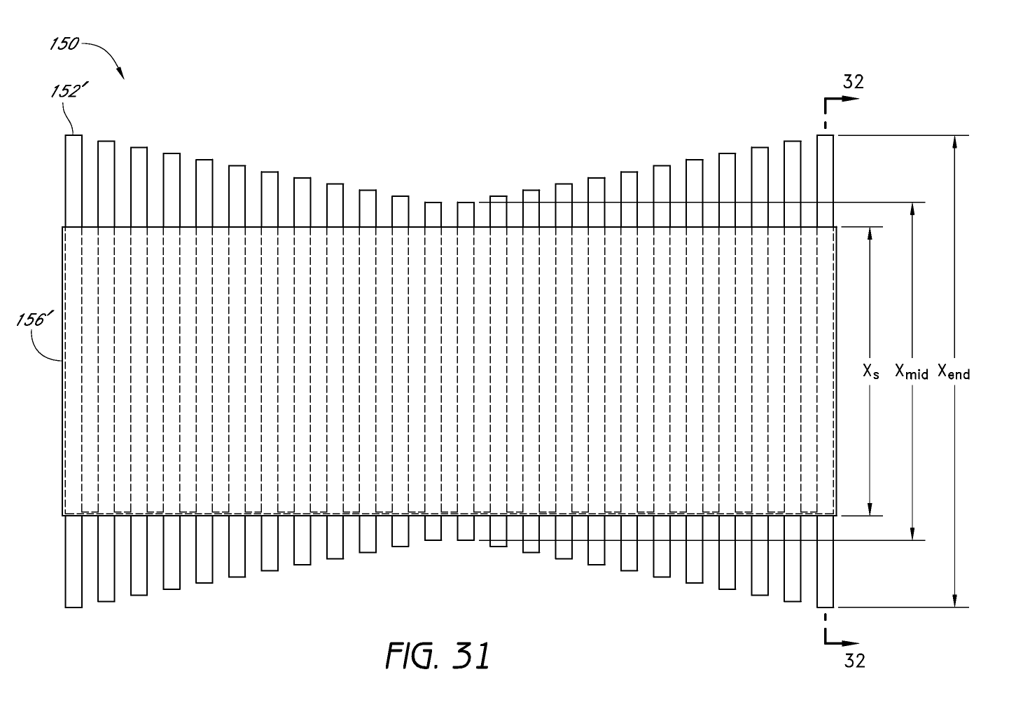

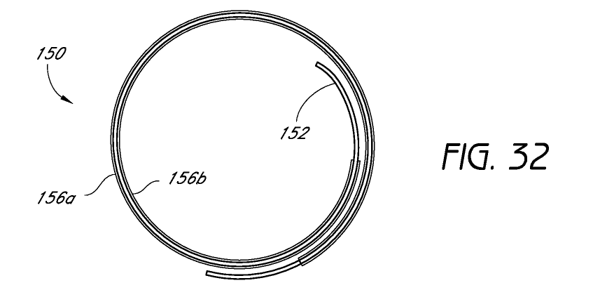

FIG. 31 is a top view of another embodiment of a stent 150′, showing the stent in a partially formed state. FIG. 32 is an enlarged section view of the embodiment of the stent 150 shown in FIG. 31 taken along line 32-32 in FIG. 31, showing the stent 150 in a rolled or formed state. In the partially formed state, as shown in FIG. 31, the material comprising the stent 150′ has been formed to the desired pre-formed size and shape, such as by, without limitation, cutting the desired size and shape from a flat sheet of material by any suitable method, including, without limitation, extrusion, chemical etching, stamping, laser cutting, water jet cutting, or by any other suitable method or operation. Additionally, in some embodiments, any of the features, dimensional and/or geometric configurations, or other details described or illustrated in this disclosure or known in the art can be applied to the stent 150.

In some embodiments (not illustrated), the stent 150 can be formed so that the interior end portion of one or more of the annular members 152 is curved inwardly. In this configuration, the inwardly curved interior end portion can improve the ability of the interior end portion to slide smoothly relative to the inside surface of the one or more of the annular members 152, and can potentially improve the dynamic responsiveness of the annular members 152. In some embodiments, as in the illustrated embodiment, the stent 150 can be formed so that the interior end portion of one or more of the annular members 152 is curved to match the contour of the surface against which it abuts.

In some embodiments, as in the illustrated embodiment, the stent 150′, 150 can be formed without connection members. Additionally, as illustrated in FIGS. 31-32, the stent 150′, 150 can comprise a sheath 156′, 156 which, among other functions, can help maintain the individually expandable annular members 152′, 152 in the desired orientation and spacing relative to other annular members 152′, 152 and/or body vessel. In the pre-formed state shown in FIG. 31, the sheath 156′ can be formed from a thin, flat sheet of any suitable material, such as, without limitation, a non-reactive polymeric material. In some embodiments, the sheath 156′, 156 can be formed from PTFE or ePTFE.

In some embodiments (not illustrated), the annular members 152′, 152 can be positioned between two layers of sheath material or embedded within the sheath material so that the sheath 156 is positioned between the annular members 152′, 152 and the body vessel, and also on the inside surface of the annular members 152′, 152. For example, in some embodiments, the annular members 152′, 152 can be laminated between two sheets of sheath material. In some embodiments, a single sheet of sheath material can be wrapped around the one or more of the edges of the annular members 152′, 152 and/or connection members.

In some embodiments, as is illustrated in FIGS. 31-32, the length “Xₐₘ” of some or all of the partially formed annular member 152′ of the stent 150′ can be greater than the length “Xₛ” of the sheath 156′ so that portions of the annular members 152′, 152 are not covered by the sheath 156′, 156. In some embodiments, where the sheath 156′, 156 can be configured to provide axial support to the annular members 152′, 152, as in the illustrated embodiment, the stent 150′, 150 can be configured so as to not have any connection members interconnecting the annular members 152′, 152. In some embodiments, the stent 150′, 150 can comprise connection members configured to interconnect the annular members 152′, 152, at any of the positions or having any of the sizes and/or geometries described above. As illustrated in FIGS. 31-32, the sheath 156′ can be positioned at the approximate middle of the annular members 152′.

The stents described herein can be formed from any of a variety of materials. The stents described herein can be formed from materials that have desirable or suitable characteristics for stents intended to be placed in body passages such as, without limitation, strength, flexibility, durability, robustness, and non-reactivity with biological fluids or materials. Suitable materials can include, without limitation, Nitinol, stainless steel, titanium, aluminum, gold, nickel-titanium, cobalt-chromium, cobalt-chromium-molybdenum, platinum, platinum-tungsten, platinum-nickel, platinum-rhenium, polyurethane, silicone elastomers, polytetrafluoroethylene, or any combination thereof.

By way of example and not limitation, any of the stents described herein can be formed by first forming the partially formed stent, such as is illustrated in FIGS. 4 and 8, from a sheet of desired material, such as but not limited to Nitinol. A partially formed stent can be formed by etching, laser cutting, stamping, mechanically cutting, or otherwise forming the sheet material to the desired geometric configuration using any suitable process. The partially formed stent can then be roll formed to the desired stent configuration. Additionally, grooves or other desired features can be formed in the annular members of the stent either before or after the roll forming process. Alternatively, any of the stents described herein can be formed from a hollow tube of the desired material.

The following discussion applies to any of the embodiments of the stents or annular members described herein. In the pre-placement state, any of the annular members described herein (such as, but not limited to, annular members 32, 42, 52, 62, 72, 82, 92, 102, 112, 122, 132, 142, and 152) can be flat without any curvature. As described herein, each of the annular members can form a generally cylindrical shape with the two ends of each annular member overlapping each other. In some embodiments, the diameter of the cylinder formed by each of the annular members in the pre-placement state (i.e., before the stent has been positioned within a person's vasculature) can be more than approximately 100% and less than approximately 200% of the diameter of each of the annular members 32 in the post-placement state (i.e., after the stent has been positioned within a person's vasculature).

Additionally, in some embodiments of any of the annular members disclosed herein, the overlapping portion can exceed 180 degrees. For example, the overlapping portion in any of the annular members can form a complete circle or revolution around the annular member, or more than one complete circle or revolution around the annular member, such as two, three, four, or more complete revolutions around the annular member.

In general, the optimum number of overlapping layers or fractional layers in the implanted, expanded configuration of the stent will depend upon a variety of factors. For example, some stents can be configured to have a sufficient overlapping surface area to resist collapse of the annular members under a radially inwardly directed pressure from an artery. Factors such as sheet thickness, spring force, and effects of various coatings upon the coefficient of static friction may affect the minimum area or length of overlap necessary to resist collapse. Although increased overlapping layers (e.g. 3 or 4 or 5 or more) can provide increased radial strength, increased overlapping may prohibit the use of a balloon to post-dilate or size the stent following initial deployment. For applications in which post deployment dilatation is desired, relatively fewer layers can be used. Thus, overlap on the order of ½ layer, one full layer, 1½ layers, 2 layers, or even 2½ layers or more may be desirable for stents intended for post deployment dilation. Additional considerations which affect the optimum overlap are disclosed elsewhere herein or in the material incorporated by reference herein. In general, for any intended implanted diameter, sheet thickness, and surface material, the optimum number of overlaps for a target artery size can be readily determined through routine experimentation by those of skill in the art in view of the disclosure herein.

Any of the stents disclosed herein can have any of the features, dimensions, components, methods, materials, or other aspects of any of the stents or other components disclosed in U.S. Pat. No. 5,728,150 (titled “Expandable Microporous Prosthesis”), issued on Mar. 17, 1998, U.S. Pat. No. Re. 35,988 (titled “Stent Construction Of Rolled Configuration”), reissued on Dec. 8, 1998, U.S. Pat. No. 6,090,136 (titled “Self Expandable Tubular Support”), issued on Jul. 18, 2000, or U.S. Pat. No. 6,120,535 (titled “Microporous Tubular Prosthesis”), issued on Sep. 19, 2000, which are all hereby incorporated by reference as if fully set forth herein.

Additionally, in some embodiments, the radius of curvature of one or both ends of one or more of the annular members described herein (such as, but not limited to, annular members 32, 42, 52, 62, 72, 82, 92, 102, 112, 122, 132, 142, and 152) comprising a stent can be smaller than the radius of curvature of the mid-section of the respective annular member. A smaller radius of curvature of one or both of the ends of the annular members can improve the coaptation of the overlapping segments of the annular members by causing at least the end of the outermost portion of the overlapping segment of the annular member to make contact with or impinge against the outside surface of the inside, overlapped portion of the annular member.

The stent can be coated with a desired coating such as polyurethane, silicone elastomers, polytetrafluoroethylene, nylon, Teflon, or other polymer or suitable material either before or after the roll forming process, depending on the desired configuration of the stent. Accordingly, the desired coating can be applied to the partially formed stent so as to be overlapping on the annular members. Further, the desired coating can be applied to either the inside surface, outside surface, or both surfaces of the annular members of the post roll formed stent. Alternatively, a thin flexible tube of suitable graft of sheath material can be formed over the outside of the stent, as illustrated in FIGS. 22-24. Additionally, the stent can be completely enveloped within the desired sheath such that the sheath material covers the inside and outside of the entire length of the stent.

Various methods can be used for delivery and implantation of a stent. The embodiments of stents described herein can be positioned in the desired location of the body passage in any method that is known in the art. For example, access to the desired body passage can be gained through the femoral artery in the leg. Or, less commonly, access to the desired body passage is gained through the radial artery or brachial artery in the arm. Once access into the artery is gained, an instrument known as a sheath introducer can be placed in the opening to keep the artery open and control bleeding. Through this sheath, a long, flexible, soft plastic tube commonly called a guiding catheter can be pushed. The guiding catheter can also allow for radiopaque dyes (commonly iodine based) to be injected into the coronary artery, so that the location of the diseased or injured body passage can be assessed using real time x-ray visualization. During the x-ray visualization, the cardiologist or surgeon can estimate the size of the body passage and can select the type of catheter and the configuration of the stent that is most suitable for the body passage.

A guidewire, which can be a thin wire that can have a radiopaque flexible tip, can be inserted into through the guiding catheter and into the body passage. The cardiologist guides the guidewire through the body passage to the site of the diseased or occluded body passage. The tip of the guidewire can be then inserted past the occluded portion of the body passage. The guidewire serves as the pathway to the occluded portion of the body passage for the stent delivery catheter. The tip of the stent delivery catheter can be hollow and can be inserted at the back of the guidewire, and envelopes the guidewire as it is inserted into the body passage. The catheter can be gently pushed forward, until the stent is located in the desired position with respect to the occluded portion of the body passage.

In one stent delivery apparatus, a self-expanding stent such as is described above can be positioned at the distal end of a catheter around a core lumen. Self-expanding stents can be held in a constricted state during delivery using a variety of methods including sheaths or sleeves which cover all or a portion of the stent. When the stent is in the desired location, i.e., in the desired position within the body passage, the sheath or sleeve can retracted to expose the stent. The stent can be configured to self-expand upon retraction of the sheath or sleeve.

Another method includes mounting a mechanically expandable stent on an expandable member, such as a dilatation balloon, provided on the distal end of an intravascular catheter. The catheter can then be advanced through a patient's vasculature to the desired location within the patient's body lumen, and the balloon can be inflated to expand the stent into a permanent expanded condition. Any of the stents illustrated in FIGS. 19-24 (or any other stents disclosed herein) can be configured to be suitable for this procedure. After the stent is in the desired position, the balloon, or other expandable member, can then be deflated and the catheter removed from the body passage. The expanded stent remains in the body passage to hold the passage open.

Although this invention has been disclosed in the context of a preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It can be also contemplated that various combinations or subcombinations of the specific features and aspects of the embodiments can be made and still fall within the scope of the invention. Accordingly, it should be understood that various features and aspects of the disclosed embodiments can be combine with or substituted for one another in order to form varying modes of the disclosed invention. Thus, it can be intended that the scope of the present invention herein disclosed should not be limited by the particular disclosed embodiments described above.

What is claimed is:

1. A prosthesis configured to support or expand a body passage, the prosthesis comprising:
a plurality of annular members arranged in series along a longitudinal axis of the prosthesis such that each annular member is longitudinally adjacent another annular member, each annular member having a generally annular shape about the longitudinal axis, each annular member defining a first end and a second opposite end, an inside surface extending between the first end and the second end, and an outside surface extending between the first end and the second end, each annular member wrapped around the longitudinal axis to form an overlapping portion where a portion of the inside surface of the annular member is adjacent to a portion of the outside surface of the respective annular member to define a longitudinal lumen having openings at a distal end of the prosthesis and at a proximal end of the prosthesis; each annular member being configured to expand and contract independent of the other annular members as the first end of an annular member moves towards the second end of the annular member and a length of the overlapping portion decreases; and
a graft extending between the plurality of annular members.

2. The prosthesis of claim 1, wherein the prosthesis is substantially cylindrically shaped in a relaxed state.

3. The prosthesis of claim 1, wherein one or more of the annular members are formed from a generally flat sheet of material.

4. The prosthesis of claim 1, further comprising a means to secure one or more of the annular members to the graft.

5. The prosthesis of claim 1, wherein one or more of the annular members are secured to the graft using adhesive.

6. The prosthesis of claim 1, wherein one or more of the annular members are secured to the graft using sutures.

7. The prosthesis of claim 1, wherein the graft is formed from a generally flat sheet of material.

8. The prosthesis of claim 1, wherein the graft is configured to cover only a portion of one or more of the annular members.

9. The prosthesis of claim 1, wherein the graft is positioned adjacent to at least a portion of the outside surface of one or more of the annular members.

10. The prosthesis of claim 1, wherein the graft is positioned adjacent to at least a portion of the inside surface of one or more of the annular members.

11. The prosthesis of claim 1, wherein one or more of the annular members is embedded within the material forming the graft or positioned between two layers of the material forming the graft.

12. The prosthesis of claim 1, further comprising one or more connection members configured to interconnect one or more of the plurality of annular members.

13. The prosthesis of claim 1, wherein one or more of the annular members are formed from a material selected from the group consisting of: Nitinol, stainless steel, titanium, aluminum, gold, nickel-titanium, cobalt-chromium, cobalt-chromium-molybdenum, platinum, platinum-tungsten, platinum-nickel, and platinum-rhenium.

14. The prosthesis of claim 1, wherein the graft is formed from a material selected from the group consisting of: urethane, Teflon, PTFE, ePTFE, polyurethane, silicone elastomers, and polytetrafluoroethylene.

15. The prosthesis of claim 1, wherein the prosthesis comprises a coating on at least one or more surfaces of the annular members.

16. The prosthesis of claim 15, wherein the coating comprises at least one material selected from the group consisting of: polyurethane, silicone elastomer, polytetrafluoroethylene, nylon, and Teflon.

17. The prosthesis of claim 1, wherein one or more of the plurality of annular members is configured to promote tissue growth into at least a portion thereof.

18. The prosthesis of claim 1, wherein one or more of the plurality of annular members is configured to be selectively biased at one or more desired diameters.

19. A method of making a prosthesis configured to support or expand a body passage, comprising:
providing a first generally flat sheet of material suitable for a graft;
providing a plurality of flat strips of material suitable for a stent;
positioning the plurality of strips in a lengthwise orientation so that each of the strips is generally parallel to one another; and
rolling the strips simultaneously with the first sheet so that each strip and the first sheet form an approximately cylindrically shaped prosthesis in which a portion of each of the strips overlap each other to define an axially extending lumen.

20. The prosthesis of claim 19, wherein the strips of material are interconnected.

21. The prosthesis of claim 19, wherein one or more of the strips are attached to the first sheet.

22. The prosthesis of claim 19, wherein one or more of the strips are attached to the first sheet using adhesive.

23. The prosthesis of claim 19, wherein one or more of the strips are attached to the first sheet using one or more sutures.

24. The prosthesis of claim 19, wherein one or more of the strips are embedded within the first sheet.

25. The prosthesis of claim 19, further comprising providing a second generally flat sheet of material suitable for a graft and melting the second sheet to the first sheet with strips being positioned generally between the first and second sheets.

26. The prosthesis of claim 19, comprising positioning the flat sheet adjacent to a first side of each of the strips, providing a second generally flat sheet of material suitable for a graft, positioning the second sheet adjacent to a second side of each of the strips, the second side opposing the first side, and then forming the strips simultaneously with the first and second sheets so that each strip and the first and second sheets form an approximately cylindrically shaped prosthesis.

27. The prosthesis of claim 19, wherein one or more of the strips define a space therebetween.

28. The prosthesis of claim 19, wherein one or more of the strips formed to define an inside surface, an outside surface, and an overlapping portion, and the inside surface of the overlapping portion of the one or more formed strips is adjacent to the outside surface of the respective annular member.

29. The prosthesis of claim 19, wherein one or more of the plurality of annular members is configured to promote tissue growth into at least a portion thereof.
